(19) United States
(12) Patent Application Publication
(10) Pub. No.: US 2013/0022629 A1
(43) Pub. Date: Jan. 24, 2013

(54) Modulators of Immunoinhibitory Receptor PD-1, and Methods of Use Thereof

(71) Applicants: Arlene H. Sharpe, Boston, MA (US); Manish J. Butte, Stanford, CA (US); Shinji Oyama, Revere, MA (US)

(72) Inventors: Arlene H. Sharpe, Boston, MA (US); Manish J. Butte, Stanford, CA (US); Shinji Oyama, Revere, MA (US)

(21) Appl. No.: 13/519,621

(22) PCT Filed: Jan. 3, 2011
(86) PCT No.: PCT/US2011/020046

§ 371 Date: Oct. 9, 2012

Related U.S. Application Data
(60) Provisional application No. 61/292,020, filed on Jan. 4, 2010.

(51) Int. Cl. A61K 31/505; A61K 31/4439; A61K 31/44; A61K 31/433; C07D 239/42; A61P 41/00; C07D 211/98; C07D 285/135; A61P 31/00; A61P 35/00; A61P 37/02; A61K 39/00; C07D 417/12

(52) U.S. Cl. 424/184.1; 514/256; 514/272; 514/342; 514/349; 514/363; 544/297; 544/327; 546/268.7; 546/297; 548/138

(57) ABSTRACT

Disclosed are an assay to identify modulators of the PD-1:PD-L pathway and PD-1:PD-L pathway modulators, e.g., compounds and pharmaceutical compositions thereof. Methods for treating diseases influenced by modulation of the PD-1:PD-L pathway such as, for example, autoimmune diseases, inflammatory disorders, allergies, transplant rejection, cancer, immune deficiency, and other immune system-related disorders, are also disclosed.

### RELATED APPLICATIONS

This application claims the benefit of priority to United States Provisional Patent Application Ser. No. 61/292,020, filed Jan. 4, 2010; the contents of which are hereby incorporated by reference.

### BACKGROUND

The programmed death PD-1 receptor and PD-L1 ligand pathway is part of the B7:CD28 family of co-stimulatory pathways. Ligation of PD-1 by either of its ligands, PD-L1 and PD-L2, during TCR signaling inhibits TCR-mediated proliferation and cytokine production (34-38). Although PD-1 is inducibly expressed on T cells following their activation, the effects of PD-1 ligation on T cells can be seen as early as 2 hrs after activation (39). It has also been shown that PD-1 can inhibit primary and secondary T cell responses (40, 41).

Interest in the PD-1:PD-L pathway has grown with the discovery that it can regulate the balance between stimulatory and inhibitory signals that regulate and maintain peripheral tolerance and the control of antimicrobial immunity. The induction and maintenance of T cell tolerance requires the PD-1 receptor and its ligands PD-L1 and PD-L2. This pathway regulates several tolerance checkpoints. First, this pathway regulates the initial decision between T cell activation versus anergy. Second, there is a limiting of the effector T cell responses. Third, nonhematopoietic expression of PD-L1 controls T cells response. In addition, this pathway also controls the development, maintenance and function of induced regulatory T cells. In turn, this will protect tissues from auto-immune damage. This pathway also has a role in autoimmune diseases and chronic infections.

Antibodies have been used to modulate the PD-1:PD-L pathway. For example, US Patent Application No. 2009/0217401 (Korman; Alan J. et al.), hereby incorporated by reference in its entriety, describes isolated monoclonal antibodies, particularly human monoclonal antibodies, that specifically bind to PD-1 with high affinity.

### SUMMARY

One aspect of the present invention relates to compounds that selectively modulate the activity of the immunoinhibitory receptor PD-1. For example, one aspect of the invention relates to a compound of formula I:

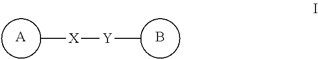

or a pharmaceutically acceptable salt, solvate, enantiomer or stereoisomer thereof, wherein independently for each occurrence,

A is aryl, heteroaryl or biaryl;

B is aryl, heteroaryl or biaryl;

X is —N(R)—, —C(R)₂—, —O— or —S—;

R is hydrogen or alkyl; and

Y is —S(═O)₂—, —S(═O)— or —C(═O)—.

Another aspect of the invention relates to a pharmaceutical composition comprising a compound of the invention (e.g., a compound of formula I, or a pharmaceutically acceptable salt, solvate, enantiomer or stereoisomer thereof), and one or more pharmaceutically acceptable carriers. A pharmaceutical composition of the invention may also comprise a second therapeutic agent. Such pharmaceutical compositions of the invention can be administered in accordance with a method of the invention (for example, as part of a therapeutic regimen for treatment or prevention of autoimmune diseases or infections). In one embodiment, the invention relates to a packaged pharmaceutical comprising a therapeutically effective amount of the compound or composition. In one embodiment, the invention relates to a packaged pharmaceutical comprising a prophylactically effective amount of the compound or composition.

Another aspect of the invention relates to a method of modulating the PD-1:PD-L pathway in a cell comprising the step of contacting a cell with a compound of the present invention, or administering a compound of the present invention to a subject in need thereof. Such methods can be used to ameliorate any condition, such as an autoimmune disease, transplant rejection, infectious diseases and/or cancer, which is caused by or potentiated by the activity of the PD-1:PD-L pathway.

Another aspect of the invention relates to a method of treating or preventing specific disorders in which the immunoinhibitory receptor PD-1 plays a part, for example, in autoimmune diseases, graft rejection, infections and/or cancer. Modulation of this pathway can be used, for example, as immunotherapy and/or for treating or preventing autoimmune diseases, graft rejection, infections and/or cancer. In certain embodiments, such methods comprise the step of administering to a subject in need thereof a therapeutically effective amount of a compound or pharmaceutical composition of the invention.

### BRIEF DESCRIPTION OF THE FIGURES

FIG. 1 depicts schema of potential effects of candidate molecules.

FIG. 2 depicts the screening results for sulfamonomethoxine and sulfamethizole, showing rescue of IFN-γ production above the negative control wells on the plate.

FIG. 3 depicts the results of validation testing of sulfamonomethoxine and sulfamethizole using wild type, PD-1^{C}, and PD-1^{-/-} T cells. Responses of PD-1^{C} and PD-1^{-/-} (KO) T cells over a range of compound concentrations (0 to 10,000 nM) are shown. Specificity is shown by block of PD-1 mediated inhibition in PD-1 Tg cells only when PD-L2 was present. Furthermore, there was no effect on PD-1^{-/-} T cells. Specific effects are seen above 400 nM in rescuing T cells from PD-1 mediated inhibition of IFN-γ production.

FIG. 4 depicts the results of structure-activity relationship testing of a compound of the invention (top). In the middle graph, the KO data are represented by the darker, flat line; Tg data are represented by the lighter, variable, top line. In the bottom graph, the KO data are represented by the darker, top, flat line; Tg data are represented by the lighter, bottom line. Error bars indicate ±1 standard deviation.

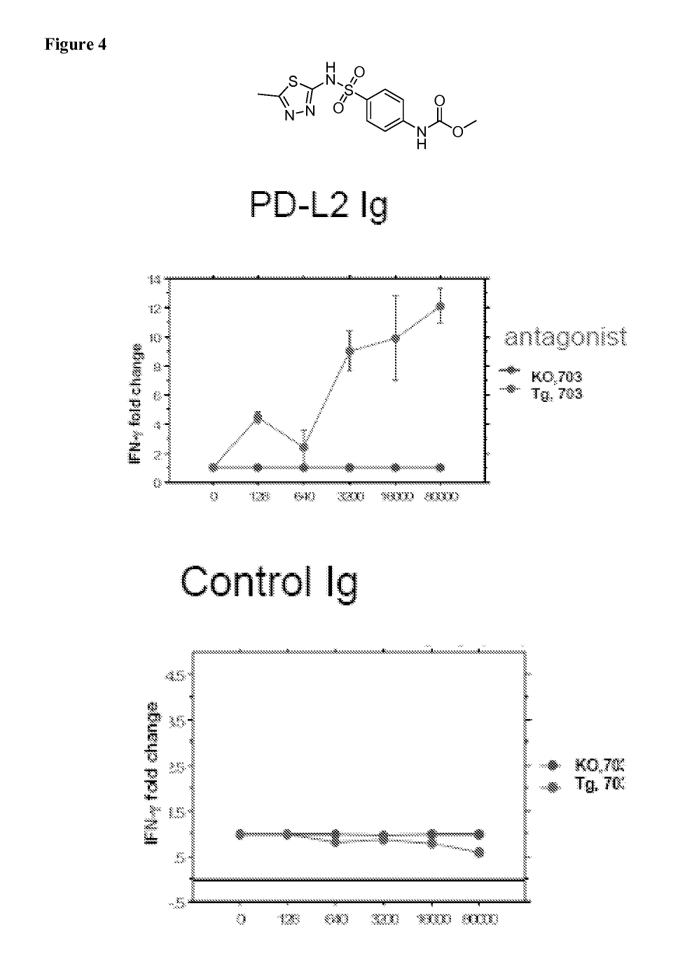

FIG. 5 depicts the results of structure-activity relationship testing of a compound of the invention (top). In the middle graph, the KO data are represented by the darker, flat line; Tg data are represented by the lighter, variable, top line. In the bottom graph, the KO data are represented by the darker, flat line; Tg data are represented by the lighter, slightly variable line. Error bars indicate ±1 standard deviation.

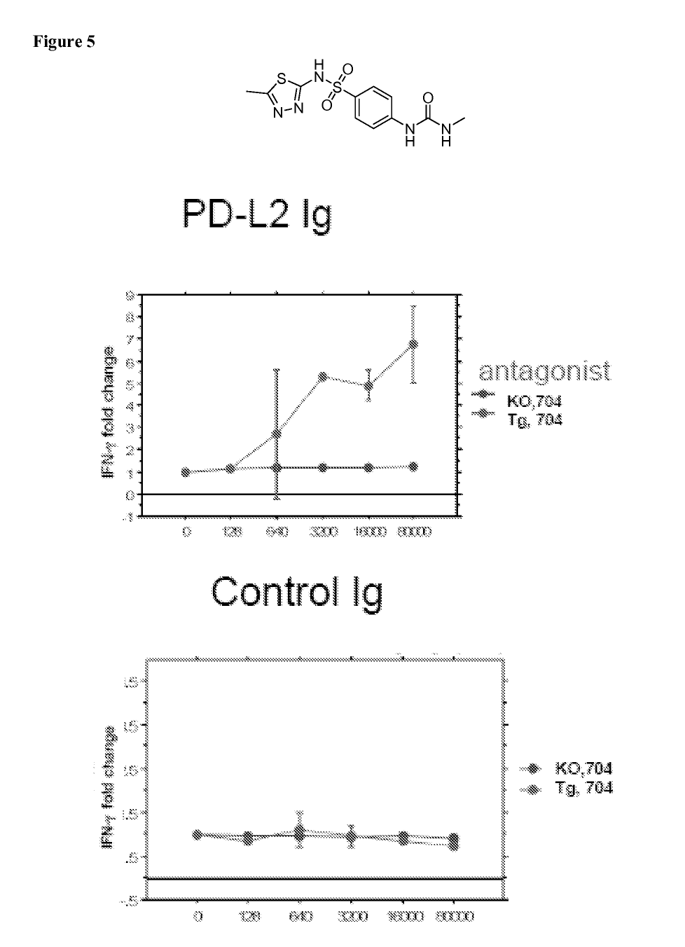

FIG. 6 depicts the results of structure-activity relationship testing of a compound of the invention (top). In the middle graph, the KO data are represented by the darker, flat line; Tg data are represented by the lighter, variable, top line. In the bottom graph, the KO data are represented by the darker, flat line; Tg data are represented by the lighter, slightly variable line. Error bars indicate ±1 standard deviation.

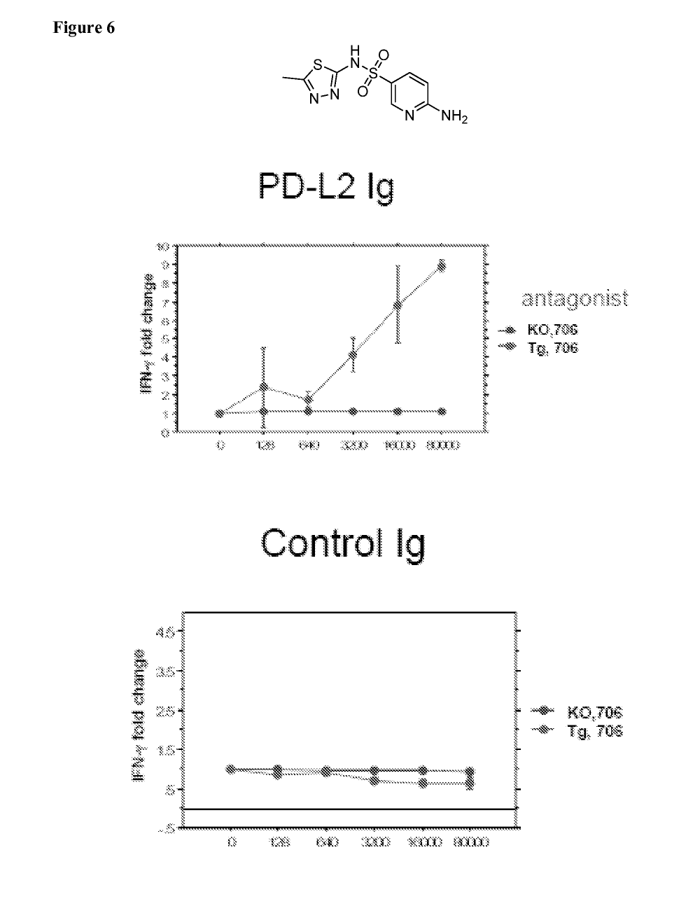

### DETAILED DESCRIPTION

One aspect of the invention relates to compounds that block the function of a key immune inhibitory pathway that restrains anti-microbial immune responses during chronic infection. This pathway, consisting of the immunoinhibitory receptor PD-1 and its two ligands PD-L1 and PD-L2, has become an attractive therapeutic target because it has a significant role in controlling the responses of T lymphocytes in a number of infectious diseases for which no cures are yet available. PD-1 mediated inhibitory signals downregulate T cell responses and facilitate microbial persistence. Modulation of this pathway during chronic viral infection can reinvigorate virus-specific T cells and lead to pathogen control in animal models. For example, the PD-1:PD-L pathway inhibits T cell responses in several emerging/re-emerging human infectious diseases (including Hepatitis C, Helicobacter pylori). In addition, this inhibitory pathway restrains anti-tumor immune responses. Specifically, expression of PD-1 ligands on tumor cells has been shown to inhibit T cell tumor immunity in animal models and high PD-L1 expression on tumors in humans is associated with poor prognosis. The PD-1:PD-L pathway also controls self-reactive T cells and protects against autoimmune diseases. In addition, the pathway regulates graft reactive T cells following organ transplantation. It follows that antagonists of the PD-1:PD-L pathway can be used to enhance anti-microbial and anti-tumor immune responses, and agonists of the PD-1:PD-L pathway can be used to treat autoimmune diseases and graft rejection.

### Screening of Compounds

In vitro assays to test for compounds that interfere with the PD-1 function have been developed. These assays utilize unique mouse strains to specifically identify PD-1-specific effects. For example, a transgenic mouse that constitutively expresses PD-1 on T cells (PD-1^{C}) has been developed (43). It has recently been disclosed that PD-1^{C} T cells are very susceptible to signaling through PD-1, as measured by a decrease in T cell proliferation and cytokine production. These PD-1^{C} T cells can be used to assess the functional effects of small molecules. A PD-1 deficient mouse (PD-1^{-/-}) has also been developed (44), and T cells from such mice will serve as useful controls for in vitro as well as in vivo studies.

In certain embodiments, PD-1^{C} T cells are cultured with a proliferative stimulus (anti-CD3) plus either PD-L2-Ig or a negative control Ig fusion protein. PD-1^{C} T cells proliferate less and produce lower amounts of cytokines when cultured with PD-L2-Ig as compared to the negative control Ig fusion protein because PD-1-ligands deliver an inhibitory signal through PD-1. Compounds of interest will modulate this inhibitory interaction in wild type and PD-1^{C} T cells, but not in PD-1^{-/-} cells. Thus, compounds which are antagonists will block PD-1 inhibitory function and lead to increased T cell responses by wild type and PD-1^{C} T cells, but will not affect PD-1^{-/-} T cells. Conversely, compounds which are agonists will deliver an inhibitory signal, and reduce T cell responses by wild type and PD-1^{C} T cells, but not PD-1^{-/-} T cells.

Because it is believed that PD-1 can inhibit cytokine (IFN-γ) production to a greater extent than T cell proliferation in a number of situations (36, 44), in certain embodiments IFN-γ production was chosen as the readout, using a bead-based fluorescent ELISA assay. These beads have a broad dynamic range (about 1 pg/mL to about 5 ng/mL), robust detection of cytokines from complex solutions (including culture media), minimal time requirement (about 3 hours from start to finish), and homogeneous format (no washing required).

Thus, for an initial screen of test compounds, one can culture PD-1^{C} T cells with plate-bound anti-CD3, PD-L2Ig (or control Ig) plus test compounds in a 384 well format assay. Tissue culture supernatants can then be assayed for IFN-γ production using beads together with high throughput flow cytometry. PD-1^{C} T cells proliferate less and produce lower amounts of cytokines when cultured with PDL2-Ig as compared to the negative control Ig fusion protein because PD-L2 delivers an inhibitory signal through PD-1.

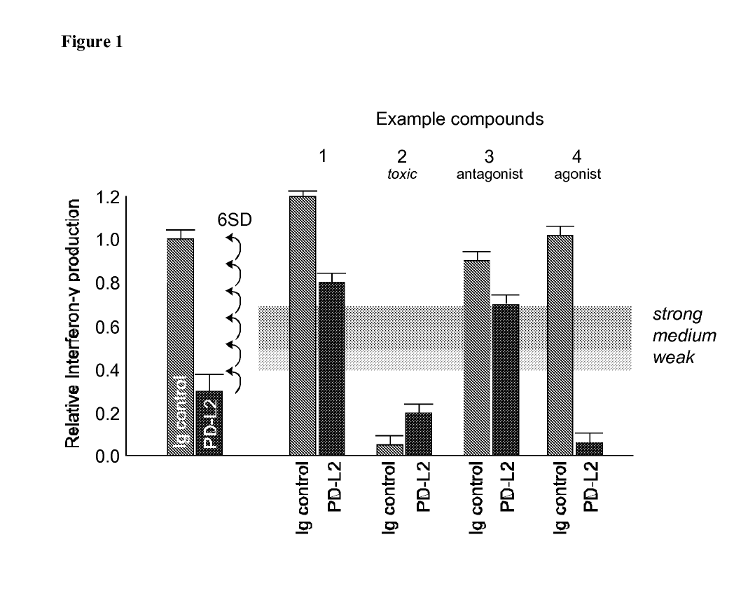

A compound was considered to be potential antagonist if it blocked PD-1 inhibitory effects (FIG. 1, compound 3). Such an antagonist leads to increased T cell function in the presence of PD-L2Ig as compared to control Ig fusion protein. Toxic compounds will decrease T cell responses in the presence of 1 g control or PD-L2Ig (FIG. 1, compound 2). Compounds that generally increase IFN-γ regardless of the PD-L2:PD-1 interaction, were eliminated as well (FIG. 1, compound 1).

A compound was considered to be a potential agonist if it reduced T cell function in the presence of PD-L2Ig relative to controls (FIG. 1, compound 4). In certain embodiments, cut offs for screening-positive hits were set as an effect that is greater than 3 standard deviations from control (e.g. in FIG. 1, compound 3 would be a screening positive for a potential antagonist).

Any compound of interest can be screened according to the present invention. Suitable test compounds include small organic compounds. Small organic compounds include a wide variety of organic molecules, such as sulfonamides, heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

### Compounds

One aspect of the invention relates to a compound of formula I:

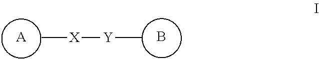

or a pharmaceutically acceptable salt, solvate, enantiomer or stereoisomer thereof, wherein independently for each occurrence,

A is aryl, heteroaryl or biaryl;

B is aryl, heteroaryl or biaryl;

X is —N(R)—, —C(R)₂—, —O— or —S—;

R is hydrogen or alkyl; and

Y is —S(═O)₂—, —S(═O)— or —C(═O)—.

In certain embodiments, the present invention relates to any one of the aforementioned compounds, wherein X is —N(R)—. In certain embodiments, the present invention relates to any one of the aforementioned compounds, wherein X is —N(H)—. In certain embodiments, the present invention relates to any one of the aforementioned compounds, wherein X is —N(CH₃)—. In certain embodiments, the present invention relates to any one of the aforementioned compounds, wherein X is —C(H)₂—. In certain embodiments, the present invention relates to any one of the aforementioned compounds, wherein X is —O—. In certain embodiments, the present invention relates to any one of the aforementioned compounds, wherein X is —S—.

In certain embodiments, the present invention relates to any one of the aforementioned compounds, wherein Y is —S(═O)₂—. In certain embodiments, the present invention relates to any one of the aforementioned compounds, wherein Y is —S(═O)—. In certain embodiments, the present invention relates to any one of the aforementioned compounds, wherein Y is —C(═O)—.

In certain embodiments, the present invention relates to any one of the aforementioned compounds, wherein X is —N(R)—; and Y is —S(═O)₂—. In certain embodiments, the present invention relates to any one of the aforementioned compounds, wherein X is —N(R)—; and Y is —C(═O)₂—.

In certain embodiments, the present invention relates to any one of the aforementioned compounds, wherein A is heteroaryl, arylheteroaryl or heteroarylheteroaryl.

In certain embodiments, the present invention relates to any one of the aforementioned compounds, wherein A is

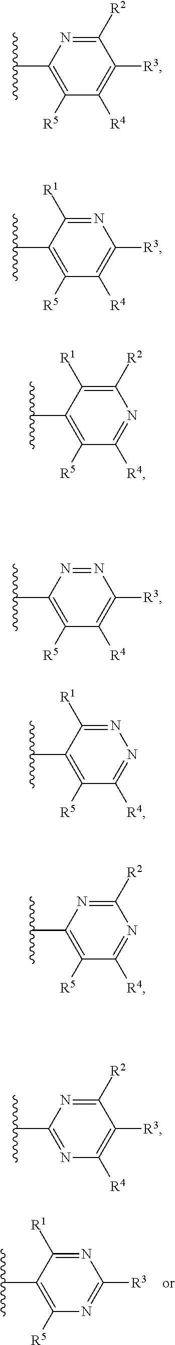

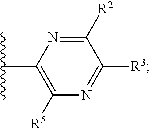

R¹ to R⁵ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

In certain embodiments, the present invention relates to any one of the aforementioned compounds, wherein R¹ is hydrogen.

In certain embodiments, the present invention relates to any one of the aforementioned compounds, wherein R² is hydrogen.

In certain embodiments, the present invention relates to any one of the aforementioned compounds, wherein R³ is hydrogen.

In certain embodiments, the present invention relates to any one of the aforementioned compounds, wherein R⁴ is hydrogen. In certain embodiments, the present invention relates to any one of the aforementioned compounds, wherein R⁴ is hydrogen, methyl, phenyl or methoxy.

In certain embodiments, the present invention relates to any one of the aforementioned compounds, wherein R⁵ is hydrogen.

In certain embodiments, the present invention relates to any one of the aforementioned compounds, wherein R¹² is hydrogen. In certain embodiments, the present invention relates to any one of the aforementioned compounds, wherein R¹² is alkyl. In certain embodiments, the present invention relates to any one of the aforementioned compounds, wherein R¹² is methyl.

In certain embodiments, the present invention relates to any one of the aforementioned compounds, wherein R¹³ is hydrogen. In certain embodiments, the present invention relates to any one of the aforementioned compounds, wherein R¹³ is alkyl. In certain embodiments, the present invention relates to any one of the aforementioned compounds, wherein R¹³ is methyl.

In certain embodiments, the present invention relates to any one of the aforementioned compounds, wherein A is

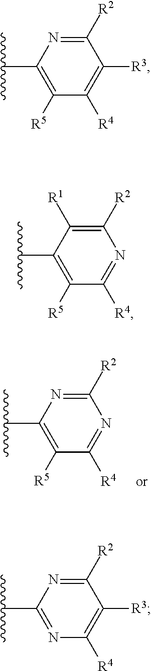

R¹ to R⁵ are independently selected from the group consisting of hydrogen, alkyl, aryl, —OR¹², —CH₂OR¹², —CH₂CH₂OR¹² and —CH₂CH₂CH₂OR¹²; and R¹² is hydrogen or alkyl.

In certain embodiments, the present invention relates to any one of the aforementioned compounds, wherein A is

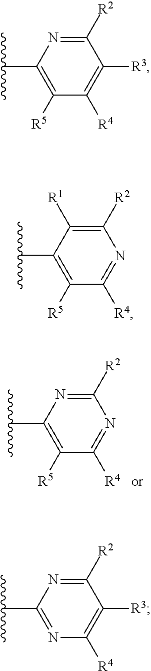

R¹, R², R³ and R⁵ are hydrogen; R⁴ is hydrogen, alkyl, aryl, —OR¹², —CH₂OR¹², —CH₂CH₂OR¹² and —CH₂CH₂CH₂OR¹²; and R¹² is hydrogen or alkyl.

In certain embodiments, the present invention relates to any one of the aforementioned compounds, wherein A is

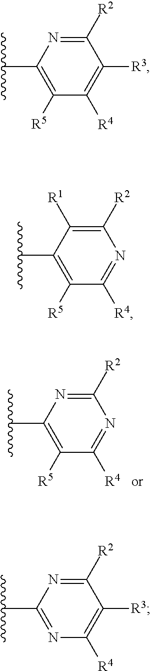

R¹, R², R³ and R⁵ are hydrogen; R⁴ is hydrogen, methyl, phenyl or methoxy.

In certain embodiments, the present invention relates to any one of the aforementioned compounds, wherein A is

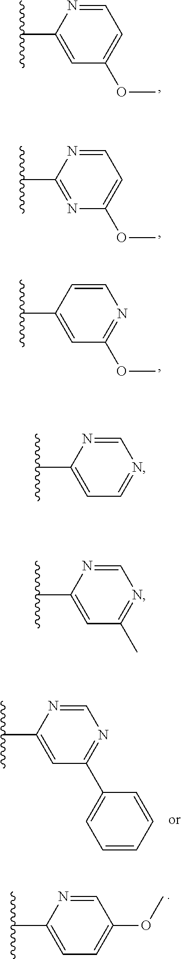

In certain embodiments, the present invention relates to any one of the aforementioned compounds, wherein A is

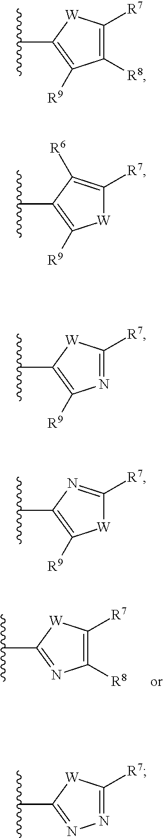

W is —S—, —O— or —N(R¹⁰)—; R⁶ to R⁹ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹⁰ is hydrogen or alkyl; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

In certain embodiments, the present invention relates to any one of the aforementioned compounds, wherein W is —S—. In certain embodiments, the present invention relates to any one of the aforementioned compounds, wherein W is —CH₂—. In certain embodiments, the present invention relates to any one of the aforementioned compounds, wherein W is —O—. In certain embodiments, the present invention relates to any one of the aforementioned compounds, wherein W is —N(R)—. In certain embodiments, the present invention relates to any one of the aforementioned compounds, wherein W is —N(H)—. In certain embodiments, the present invention relates to any one of the aforementioned compounds, wherein W is —N(CH₃)—.

In certain embodiments, the present invention relates to any one of the aforementioned compounds, wherein R¹² is hydrogen. In certain embodiments, the present invention relates to any one of the aforementioned compounds, wherein R¹² is alkyl. In certain embodiments, the present invention relates to any one of the aforementioned compounds, wherein R¹² is methyl.

In certain embodiments, the present invention relates to any one of the aforementioned compounds, wherein R¹³ is hydrogen. In certain embodiments, the present invention relates to any one of the aforementioned compounds, wherein R¹³ is alkyl. In certain embodiments, the present invention relates to any one of the aforementioned compounds, wherein R¹³ is methyl.

In certain embodiments, the present invention relates to any one of the aforementioned compounds, wherein A is

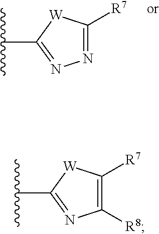

W is —S—; and R⁷ and R⁸ are independently selected from the group consisting of hydrogen, alkyl, alkyloxy, alkyloxyalkyl and aryl.

In certain embodiments, the present invention relates to any one of the aforementioned compounds, wherein A is

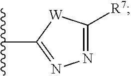

W is —S—; and R⁷ is hydrogen, alkyl, aryl, —OR¹², —CH₂OR¹², —CH₂CH₂OR¹² or —CH₂CH₂CH₂OR¹²; and R¹² is hydrogen or alkyl.

In certain embodiments, the present invention relates to any one of the aforementioned compounds, wherein A is

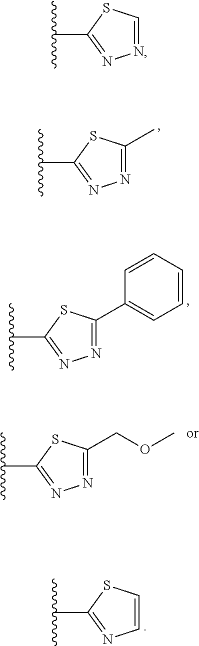

In certain embodiments, the present invention relates to any one of the aforementioned compounds, wherein B is aryl. In certain embodiments, the present invention relates to any one of the aforementioned compounds, wherein B is substituted phenyl.

In certain embodiments, the present invention relates to any one of the aforementioned compounds, wherein B is

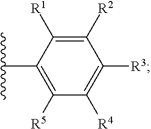

R¹ to R⁵ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²), R¹³, —CH₂CH₂CH₂N(R¹²)R¹³, —OR¹², —CH₂OR¹², —CH₂CH₂OR¹² and —CH₂CH₂CH₂OR¹²; R¹⁰ is hydrogen or alkyl; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

In certain embodiments, the present invention relates to any one of the aforementioned compounds, wherein R¹ is hydrogen.

In certain embodiments, the present invention relates to any one of the aforementioned compounds, wherein R² is hydrogen.

In certain embodiments, the present invention relates to any one of the aforementioned compounds, wherein R³ is hydrogen. In certain embodiments, the present invention relates to any one of the aforementioned compounds, wherein R³ is —N(R¹²)R¹³, —CH₂N(R¹²)R¹³, —CH₂CH₂N(R¹²)R¹³ or —CH₂CH₂CH₂N(R¹²)R¹³. In certain embodiments, the present invention relates to any one of the aforementioned compounds, wherein R³ is —N(R¹²)R¹³. In certain embodiments, the present invention relates to any one of the aforementioned compounds, wherein R³ is −OR¹², —CH₂OR¹², —CH₂CH₂OR¹² or —CH₂CH₂CH₂OR¹². In certain embodiments, the present invention relates to any one of the aforementioned compounds, wherein R³ is −OR¹².

In certain embodiments, the present invention relates to any one of the aforementioned compounds, wherein R⁴ is hydrogen.

In certain embodiments, the present invention relates to any one of the aforementioned compounds, wherein R⁵ is hydrogen.

In certain embodiments, the present invention relates to any one of the aforementioned compounds, wherein R¹² is hydrogen, alkyl, alkylcarbonyl, alkoxycarbonyl or amido. In certain embodiments, the present invention relates to any one of the aforementioned compounds, wherein R¹² is hydrogen. In certain embodiments, the present invention relates to any one of the aforementioned compounds, wherein R¹² is alkyl. In certain embodiments, the present invention relates to any one of the aforementioned compounds, wherein R¹² is methyl. In certain embodiments, the present invention relates to any one of the aforementioned compounds, wherein R¹² is methylcarbonyl. In certain embodiments, the present invention relates to any one of the aforementioned compounds, wherein R¹² is methyoxycarbonyl. In certain embodiments, the present invention relates to any one of the aforementioned compounds, wherein R¹² is methylaminocarbonyl.

In certain embodiments, the present invention relates to any one of the aforementioned compounds, wherein R¹³ is hydrogen. In certain embodiments, the present invention relates to any one of the aforementioned compounds, wherein R¹³ is alkyl. In certain embodiments, the present invention relates to any one of the aforementioned compounds, wherein R¹³ is methyl.

In certain embodiments, the present invention relates to any one of the aforementioned compounds, wherein R¹² is hydrogen; and R¹³ is hydrogen. In certain embodiments, the present invention relates to any one of the aforementioned compounds, wherein R¹² is methyl; and R¹³ is hydrogen.

In certain embodiments, the present invention relates to any one of the aforementioned compounds, wherein B is

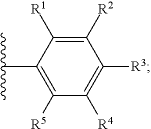

R¹, R², R⁴ and R⁵ are hydrogen; R³ is hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹⁰ is hydrogen or alkyl; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

In certain embodiments, the present invention relates to any one of the aforementioned compounds, wherein B is

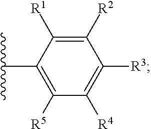

R¹, R², R⁴ and R⁵ are hydrogen; R³ is —N(R¹²)R¹³ or —OR¹²; and R¹³ is hydrogen or methyl.

In certain embodiments, the present invention relates to any one of the aforementioned compounds, wherein B is

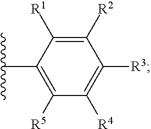

R¹, R², R⁴ and R⁵ are hydrogen; R³ is —N(R¹²)R¹³ or —OR¹²; R¹² is hydrogen, alkylcarbonyl, alkyloxycarbonyl or amido; and R¹³ is hydrogen or methyl.

In certain embodiments, the present invention relates to any one of the aforementioned compounds, wherein B is

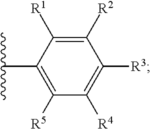

R¹, R², R⁴ and R⁵ are hydrogen;

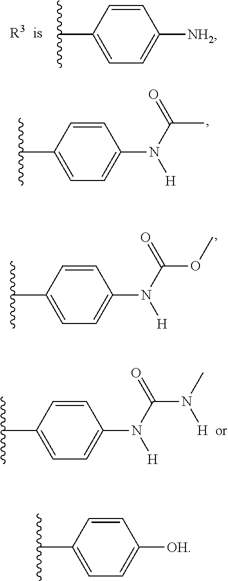

One aspect of the invention relates to a compound, or a pharmaceutically acceptable salt, solvate, enantiomer or stereoisomer thereof, selected from the group consisting of

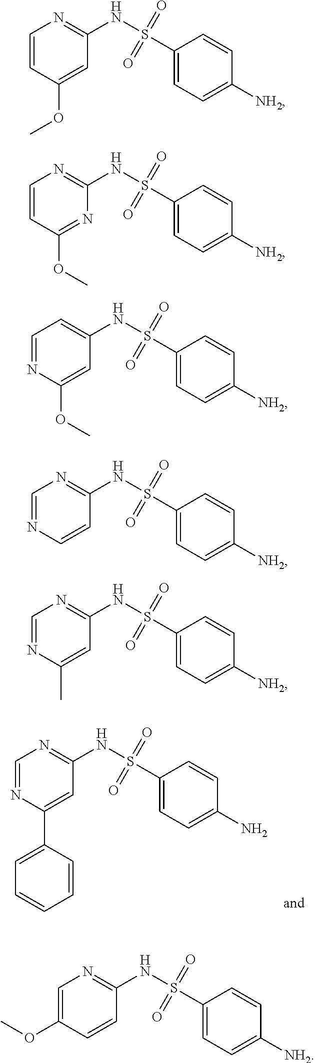

One aspect of the invention relates to a compound, or a pharmaceutically acceptable salt, solvate, enantiomer or stereoisomer thereof, selected from the group consisting of

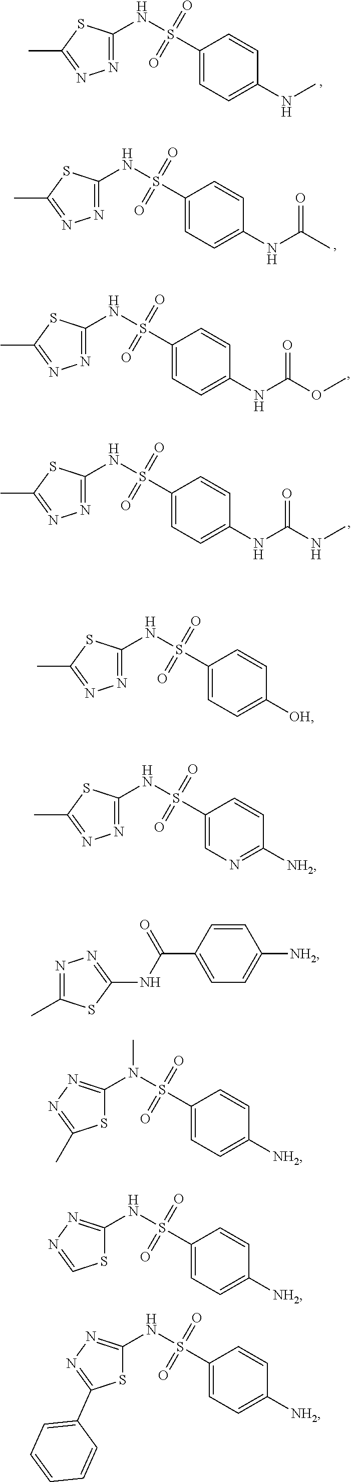

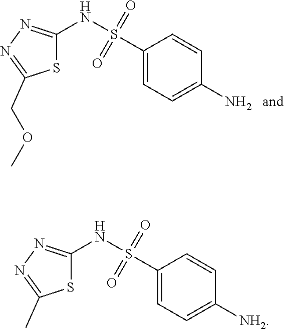

In certain embodiments, the invention relates to any one of the aforementioned compounds and attendant definitions, wherein the compound is an modulator of the PD-1:PD-L pathway.

In certain embodiments, the invention relates to any one of the aforementioned compounds and attendant definitions, wherein the compound is a PD-1 antagonist.

In certain embodiments, the invention relates to any one of the aforementioned compounds and attendant definitions, wherein the compound is a PD-1 agonist.

In certain embodiments, the invention relates to any one of the aforementioned compounds and attendant definitions, wherein the compound is a selective inhibitor of the PD-1:PD-L1 pathway.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a subject. Pharmaceutically compatible salts may be formed with many acids, including but not limited to hydrochloric, sulfuric, acetic, lactic, tartaric, malic, and succinic acids. Salts tend to be more soluble in water or other protic solvents than their corresponding free base forms. The present invention includes such salts.

Pharmaceutically acceptable acid addition salts include those formed with mineral acids such as hydrochloric acid and hydrobromic acid, and also those formed with organic acids such as maleic acid. For example, acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di alkyl-N-(hydroxy alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Certain compounds of the invention and their salts may exist in more than one crystalline form (i.e., polymorph); the present invention includes each of the crystal forms and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. When compounds of the invention contain one chiral center, the compounds exist in two enantiomeric forms and the present invention includes both enantiomers and mixtures of enantiomers, such as racemic mixtures thereof. The enantiomers may be resolved by methods known to those skilled in the art; for example, enantiomers may be resolved by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example, via enzymatic esterification; or gas-liquid or liquid chromatography in a chiral environment, for example, on a chiral support; suitable include chiral supports (e.g., silica with a bound chiral ligand) or in the presence of a chiral solvent. Where the desired enantiomer is converted into another chemical entity by one of the separation procedures described above, a further step may be used to liberate the desired purified enantiomer. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, it may exist in diastereoisomeric forms. The diastereoisomeric compounds may be separated by methods known to those skilled in the art (for example, chromatography or crystallization) and the individual enantiomers may be separated as described above. The present invention includes the various diastereoisomers of compounds of the invention, and mixtures thereof. Compounds of the invention may exist in different tautomeric forms or as different geometric isomers, and the present invention includes each tautomer and/or geometric isomer of compounds of the invention, and mixtures thereof. Compounds of the invention may exist in zwitterionic form. The present invention includes each zwitterionic form of compounds of the invention, and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs release an amine of a compound of the invention wherein the free hydrogen of an amine or alcohol is replaced by (C₁-C₆)alkanoyloxymethyl, 1-((C₁-C₆)alkanoyl-oxy)ethyl, 1-methyl-1-((C₁-C₆)alkanoyloxy)ethyl, (C₁-C₆)alkoxycarbonyl-oxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

Other exemplary pro-drugs upon cleavage release a corresponding free acid, and such hydrolyzable ester-forming residues of the compounds of this invention include but are not limited to carboxylic acid substituents (e.g., —(CH₂)C(O)OH or a moiety that contains a carboxylic acid) wherein the free hydrogen is replaced by (C₁-C₄)alkyl, (C₂-C₁₂)alkanoyloxy-methyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyl-oxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylamino-ethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

### General Synthetic Methods

Some of the general methods which were utilized to prepare the compounds disclosed in this application are described below.

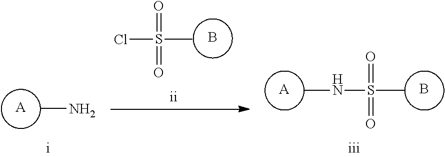

In one approach to the preparation of sulfonamides, an aryl, heteroaryl or biaryl amine (i) can be reacted with an aryl, heteroaryl or biaryl sulfonyl chloride (ii) to yield the desired sulfonamides (iii). Further chemical manipulation can then be done on any substituents on the A and/or B rings, as well as on the N—H of the sulfonamide (e.g. methylation). The synthesis of compounds other than sulfonamides, such as amides, can be effected by using a different nucleophile or electrophile, such as using an acyl chloride (—C(═O)Cl) instead of a sulfonyl chloride (ii).

In addition, it may be convenient or desirable to prepare, purify, and/or handle the active compound in a chemically protected form. The term “chemically protected form,” as used herein, pertains to a compound in which one or more reactive functional groups are protected from undesirable chemical reactions (i.e., they have been modified with a protecting group).

By protecting a reactive functional group, reactions involving other unprotected reactive functional groups can be performed without affecting the protected group; the protecting group may be removed, usually in a subsequent step, without substantially affecting the remainder of the molecule. See, for example, Protective Groups in Organic Synthesis (T. Green and P. Wuts, Wiley, 1991), and Protective Groups in Organic Synthesis (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (C(═O)) is converted to a diether (C(OR)₂), by reaction with, for example, a primary alcohol. The aldehyde or ketone group is readily regenerated by hydrolysis using a large excess of water in the presence of acid.

For example, an amine group may be protected, for example, as an amide (—NRC(═O)R) or a urethane (—NRC(═O)OR), for example, as: a methyl amide (—NHC(═O)CH₃); a benzyloxy amide (—NHC(═O)OCH₂C₆H₅NHCbz); as a t-butoxy amide (—NHC═(═O)OC(CH₃)₃, —NHBoc); a 2-biphenyl-2-propoxy amide (—NHC(═O)OC(CH₃)₂C₆H₄C₆H₅NHBoc), as a 9-fluorenylmethoxy amide (—NHFmoc), as a 6-nitroveratryloxy amide (—NHNvoc), as a 2-trimethylsilylethyloxy amide (—NHTeoc), as a 2,2,2-trichloroethyloxy amide (—NHTroc), as an allyloxy amide (—NHAlloc), as a 2-(phenylsulfonyl)ethyloxy amide (—NHPsec); or, in suitable cases (e.g., cyclic amines), as a nitroxide radical.

For example, a carboxylic acid group may be protected as an ester or an amide, for example, as: a benzyl ester; a t-butyl ester; a methyl ester; or a methyl amide.

For example, a thiol group may be protected as a thioether (—SR), for example, as: a benzyl thioether; or an acetamidomethyl ether (—SCH₂NHC(═O)CH₃).

### Pharmaceutical Compositions

One or more compounds of this invention can be administered to a human patient by themselves or in pharmaceutical compositions where they are mixed with suitable carriers or excipient(s) at doses to treat or ameliorate a disease or condition as described herein. Mixtures of these compounds can also be administered to the patient as a simple mixture or in suitable formulated pharmaceutical compositions. For example, one aspect of the invention relates to a pharmaceutical composition comprising a therapeutically effective dose of a compound of formula I, or a pharmaceutically acceptable salt, solvate, enantiomer or stereoisomer thereof; and a pharmaceutically acceptable diluent or carrier.

Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer a compound in a local rather than a systemic manner, for example, via injection of the compound directly into an edematous site, often in a depot or sustained release formulation.

Furthermore, one may administer a compound in a targeted drug delivery system, for example, in a liposome coated with endothelial-cell-specific antibody.

The pharmaceutical compositions of the present invention may be manufactured, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants are used in the formulation appropriate to the barrier to be permeated. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for reconstitution before use with a suitable vehicle, e.g., sterile pyrogen-free water.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives (for example, as a sparingly soluble salt).

Alternatively, other delivery systems for hydrophobic pharmaceutical compounds may be employed. Liposomes and emulsions are examples of delivery vehicles or carriers for hydrophobic drugs. Certain organic solvents such as dimethysulfoxide also may be employed. Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers, such as polyethylene glycols.

### Combination Therapy

In one aspect of the invention, a compound of the invention, or a pharmaceutically acceptable salt thereof, can be used in combination with another therapeutic agent to treat diseases such cancer and/or neurological disorders. For example, the additional agent can be a therapeutic agent that is art-recognized as being useful to treat the disease or condition being treated by the compound of the present invention. The additional agent also can be an agent that imparts a beneficial attribute to the therapeutic composition (e.g., an agent that affects the viscosity of the composition).

The combination therapy contemplated by the invention includes, for example, administration of a compound of the invention, or a pharmaceutically acceptable salt thereof, and additional agent(s) in a single pharmaceutical formulation as well as administration of a compound of the invention, or a pharmaceutically acceptable salt thereof, and additional agent(s) in separate pharmaceutical formulations. In other words, co-administration shall mean the administration of at least two agents to a subject so as to provide the beneficial effects of the combination of both agents. For example, the agents may be administered simultaneously or sequentially over a period of time.

The agents set forth below are for illustrative purposes and are not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

For example, one aspect of the invention relates to the use of compounds of the invention (e.g., those of formula I) in combination with at least one other anti-inflammatory or immunosuppressant agent. Examples of therapeutic agents that can be used in combination therapy are described in greater detail below in the section on uses of the compounds of the invention.

### Dosage

As used herein, a “therapeutically effective amount” or “therapeutically effective dose” is an amount of a compound of the invention or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount may be determined by methods known to those of skill in the art.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

For administration of a compounds of the invention, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a compound of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

### Kits

The compounds and compositions of the invention (e.g., compounds and compositions of formula I) may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition. Instructions for use may also be provided.

### Uses and Methods

As noted above, PD-1 is an immunoreceptor belonging to the CD28/CTLA-4 family which negatively regulates antigen receptor signaling by mechanisms that include recruitment of protein tyrosine phosphatases, SHP-1 or SHP-2, upon interacting with either of two ligands, PD-L1 or PD-L2. Because of the wide range of ligand distribution in the body, its biological significance pervades almost every aspect of immune responses including autoimmunity, tumor immunity, infectious immunity, transplantation immunity, allergy and immunological privilege.

The compounds, compositions and methods of the present invention have numerous in vitro and in vivo utilities involving, for example, detection of PD-1 or enhancement of immune response by modulation of PD-1. In certain embodiments, the compounds of the invention can be administered to cells in culture, in vitro or ex vivo, or to subjects, e.g., in vivo, to enhance immunity in a variety of situations. Accordingly, in one aspect, the invention provides a method of modifying an immune response in a subject comprising administering to the subject a compound of the invention such that the immune response in the subject is modified.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. In certain embodiments, subjects include human patients in need of enhancement of an immune response. The methods are suitable for treating human patients having a disorder that can be treated by augmenting the T-cell mediated immune response. In certain embodiments, the methods are suitable for treatment of cancer cells in vivo. To achieve antigen-specific enhancement of immunity, the compounds of the invention can be administered together with an antigen of interest. When the compounds of the invention are administered together with another agent, they can be administered in any order or simultaneously.

The invention further provides methods for detecting the presence of human PD-1 in a sample, or measuring the amount of human PD-1, comprising contacting the sample, and a control sample, with a compound of the invention which specifically binds to human PD-1, under conditions that allow for formation of a complex between the compound and human PD-1. The formation of a complex is then detected, wherein a difference in complex formation between the sample compared to the control sample is indicative of the presence of human PD-1 in the sample.

In certain embodiments, the agonists of the invention can be used in circumstances where a reduction in the level of immune response may be desirable, for example, in certain types of allergy or allergic reactions (e.g., by inhibition of IgE production), autoimmune diseases (e.g., rheumatoid arthritis, type I diabetes mellitus, multiple sclerosis, inflammatory bowel disease, Crohn's disease, and systemic lupus erythematosis), tissue, skin and organ transplant rejection, and graft-versus-host disease (GVHD). In addition, under certain circumstances it may be desirable to elicit or enhance a patient's immune response in order to treat an immune disorder or cancer. The disorders being treated or prevented by the disclosed methods include but are not limited to infections with microbes (e.g. bacteria), viruses (e.g., systemic viral infections such as influenza, viral skin diseases such as herpes or shingles), or parasites; and cancer (e.g., melanoma and prostate cancers).

In addition, under certain circumstances it may be desirable to elicit or enhance a patient's immune response in order to treat an immune disorder or cancer. The disorders being treated or prevented by the disclosed methods include but are not limited to infections with microbes (e.g. bacteria), viruses (e.g., systemic viral infections such as influenza, viral skin diseases such as herpes or shingles), or parasites; and cancer (e.g., melanoma and prostate cancers).

In certain embodiments, stimulation of immune responses with compounds of the invention which are antagonists of PD-1 enhances T cell responses. Thus, in some embodiments, compounds of the invention can be used to inhibit or reduce the downregulatory activity associated with PD-1, e.g., the activity associated with downregulation of TcR/CD28-mediated immune response. For example, modulation of PD-1/PD-L interaction with antagonizing compounds should lead to enhanced T cell proliferative responses, consistent with a downregulatory role for the PD-1 pathway in T responses.

PD-1 is also expressed on B cells, macrophages and dendritic cells. Far less is know about PD-1 function on B cells, macrophages and DC. However, PD-1 also can inhibit responses of B cells and macrophages. Therefore, PD-1 antagonist and agonist compounds have the potential to modulate responses of any PD-1 expressing cell. For example, in certain embodiments, compounds of the invention which are PD-1 antagonists may be used to enhance B cell or macrophage responses.

In certain embodiments, the compounds of the invention inhibit binding of PD-L to PD-1 with an IC₅₀ of, for example, less than 10 nM, less then 5 nM, or less than 1 nM. Inhibition of PD-L binding can be measured as described herein or using techniques known in the art.

### Cancer

Blockade of PD-1 can enhance the immune response to cancerous cells in the patient. The ligand for PD-1, PD-L1, is upregulated in a variety of human cancers. The level of PD-L1 expression correlates with prognosis: the higher the expression of PD-L1, the poorer the prognosis. The interaction between PD-1 and PD-L1 contributes to the multiple barriers that prevent anti-tumor responses, and shields tumors from immune eradication. Signals through this pathway can decrease tumor infiltrating lymphocytes, decrease T-cell receptor mediated proliferation, and induce regulatory T cells, promoting immune evasion by the cancerous cells. Immune suppression can be reversed by inhibiting the local interaction of PD-1 with PD-L1 and the effect may be additive when the interaction of PD-1 with PD-L2 is blocked as well. Previous studies have shown that T-cell proliferation can be restored by inhibiting the interaction of PD-1 with PD-L1. In one aspect, the present invention relates to treatment of a subject using a compound of the invention, which is a PD-1/PD-L1 antagonist, such that growth of cancerous tumors is inhibited. A compound of the invention may be used alone to inhibit the growth of cancerous tumors. Alternatively, a compound of the invention may be used, e.g., in conjunction with other immunogenic agents, standard cancer treatments, or antibodies.

Accordingly, in one embodiment, the invention provides a method of inhibiting growth of tumor cells in a subject, comprising administering to the subject a therapeutically effective amount of a compound of the invention.

For example, cancers whose growth may be inhibited using the compounds of the invention include cancers typically responsive to immunotherapy. Non-limiting examples of cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, the invention includes refractory or recurrent malignancies whose growth may be inhibited using the compounds of the invention.

Examples of other cancers that may be treated using the methods of the invention include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, hematopoietic cancers, such as acute myeloid leukemia (AML), and combinations of said cancers. The present invention is also useful for treatment of metastatic cancers, especially metastatic cancers that express PD-L1.

Optionally, compounds of the invention can be combined with an immunogenic agent, such as cancerous cells, purified tumor antigens (including recombinant proteins, peptides, and carbohydrate molecules), and cells transfected with genes encoding immune stimulating cytokines Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF.

In humans, some tumors have been shown to be immunogenic, such as melanomas. It is anticipated that by raising the threshold of T cell activation by PD-1 blockade, one may expect to activate tumor responses in the subject.

PD-1 blockade is likely to be most effective when combined with a vaccination protocol. Many experimental strategies for vaccination against tumors have been devised. In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination.

The study of gene expression and large scale gene expression patterns in various tumors has led to the definition of so called tumor specific antigens. In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. PD-1 blockade may be used in conjunction with a collection of recombinant proteins and/or peptides expressed in a tumor in order to generate an immune response to these proteins. These proteins are normally viewed by the immune system as self antigens and are therefore tolerant to them. The tumor antigens may also include the protein telomerase, which is required for the synthesis of telomeres of chromosomes and which is expressed in more than 85% of human cancers and in only a limited number of somatic tissues. (These somatic tissues may be protected from immune attack by various means). Tumor antigens may also be “neo-antigens” expressed in cancer cells because of somatic mutations that alter protein sequence or create fusion proteins between two unrelated sequences (e.g. bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with PD-1 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity.

Dendritic cells (DC) are potent antigen presenting cells that can be used to prime antigen-specific responses. DC's can be produced ex vivo and loaded with various protein and peptide antigens as well as tumor cell extracts. DCs may also be transduced by genetic means to express these tumor antigens as well. DCs have also been fused directly to tumor cells for the purposes of immunization. As a method of vaccination, DC immunization may be effectively combined with PD-1 blockade to activate more potent anti-tumor responses.

PD-1 blockade may also be combined with standard cancer treatments. For example, PD-1 blockade may be effectively combined with chemotherapeutic regimes. In these instances, it may be possible to reduce the dose of chemotherapeutic reagent administered. An example of such a combination is a compound of the invention in combination with decarbazine for the treatment of melanoma. Another example of such a combination is a compound of the invention in combination with interleukin-2 (IL-2) for the treatment of melanoma. The scientific rationale behind the combined use of PD-1 blockade and chemotherapy is that cell death, that is a consequence of the cytotoxic action of most chemotherapeutic compounds, should result in increased levels of tumor antigen in the antigen presentation pathway. Other combination therapies that may result in synergy with PD-1 blockade through cell death are radiation, surgery, and hormone deprivation. Each of these protocols creates a source of tumor antigen in the host. Angiogenesis inhibitors may also be combined with PD-1 blockade Inhibition of angiogenesis leads to tumor cell death, which may feed tumor antigen into host antigen presentation pathways.

PD-1 blocking compounds of the invention can also be used in combination with bispecific antibodies that target Fc alpha or Fc gamma receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses may be augmented by the use of PD-1 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-beta, IL-10, and Fas ligand. Antibodies to each of these entities may be used in combination with PD-1 antagonist compounds of the invention to counteract the effects of the immunosuppressive agent and favor tumor immune responses by the host.

Bone marrow transplantation is currently being used to treat a variety of tumors of hematopoietic origin. While graft versus host disease is a consequence of this treatment, therapeutic benefit may be obtained from graft vs. tumor responses. PD-1 blockade can be used to increase the effectiveness of the donor engrafted tumor specific T cells.

There are also several experimental treatment protocols that involve ex vivo activation and expansion of antigen specific T cells and adoptive transfer of these cells into subjects in order to antigen-specific T cells against tumor. Ex vivo activation in the presence of anti-PD-1 compounds of the invention may be expected to increase the frequency and activity of the adoptively transferred T cells.

### Infectious Diseases

Other methods of the invention are used to treat subjects that have been exposed to particular toxins or pathogens. Accordingly, another aspect of the invention provides a method of treating an infectious disease in a subject comprising administering to the subject a compound of the invention which is a PD-1 antagonist, such that the subject is treated for the infectious disease.

Progressive chronic or persistent viral infections, such as HIV or HCV, cause significant morbidity and mortality. Both CD8 and CD4 T cells are critical in the immune response to chronic viral infections and long-term control of latent and reactivating viruses. It appears that CD8 T cells are the major effectors exerting viral control and that CD4 T cell “help” is essential for sustaining CD8 T cell function during chronic infection (1, 2). Unfortunately, in many instances the host T cell response fails to effectively contain viral replication (3, 4), and such infections are associated with suppressed T cell immunity (5-7). Defining the mechanisms that lead to this loss of T cell function during chronic infections may provide new targets for therapeutic intervention and augmenting of antiviral responses. Pathways in the B7:CD28 family regulate the balance between stimulatory and inhibitory signals needed for effective immune responses to microbes, while maintaining self-tolerance (for recent reviews, see (8-11)). Recent studies indicate that the PD-1:PD-L pathway (12, 13), has a central role in regulating the interplay between host defenses aimed at eradicating pathogenic microbes and microbial strategies that evolved to resist immune responses and persist in the host (14).

For example, a number of microorganisms that cause chronic infection appear to have exploited the PD-1:PD-L pathway to evade host immune effector mechanisms and establish persistent infection (11, 13). Following an acute infection or vaccination, effective antiviral T cells acquire the ability to perform multiple effector functions upon antigen stimulation, including cytokine production, cytotoxicity, and proliferation. Chronic viral infections, in contrast, are often characterized by T cell dysfunction (1, 7). For example, during chronic LCMV infection, virus-specific CD8 T cells lose the ability to elicit effector functions in a progressive and hierarchical manner (4). T cell “exhaustion” has been observed in many chronic viral infections in both animal models and humans (including HIV, HBV, HCV) (5-7, 17, 23, 42). Thus, while the precise functional defects often differ, the general concept of T cell dysfunction appears to be a common feature of persisting infections.

The PD-1:PD-L pathway contributes directly to T cell dysfunction and lack of viral control in established chronic infection. In vivo blockade of PD-1:PD-L1 interactions in mice restored T cell function and enhanced viral control in mice with chronic LCMV infection (14). These observations were quickly extended to humans. PD-1 levels are elevated on HIV, HBV and HCV-specific T cells (16, 17, 19-22, 24-26). Blocking PD-1:PD-L interactions in vitro reversed exhaustion of HIV and HCV-specific CD8 and CD4 T cells and restored cytokine production and proliferation (16, 19-21, 26). Thus, both animal models and human studies indicate that PD-1 expression on virus-specific T cells may limit antiviral effectiveness by downregulating function and proliferation, and suggest a new therapeutic strategy for chronic viral infection. Blockade of the PD-1:PD-L pathway has the potential to reinvigorate exhausted virus-specific T cells and improve T cells responses to therapeutic vaccination, leading to enhanced viral control (15).

Similar to its application to tumors as discussed above, PD-1 blockade can be used alone, or as an adjuvant, in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas Aeruginosa. PD-1 blockade is particularly useful against established chronic or persistent infections by agents such as HIV that present altered antigens over the course of the infections. These novel epitopes are recognized as foreign at the time of anti-human PD-1 administration, thus provoking a strong T cell response that is not dampened by negative signals through PD-1.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

The PD-1:PD-L pathway appears to have a key role in chronicity of bacterial infections. For example, during H. pylori infection, T cell responses are insufficient to clear bacteria, leading to persistent infection. Following exposure to H. pylori, PD-L1 is upregulated on human gastric epithelial cells in vitro or in vivo (33). Anti-PD-L1 blocking antibodies enhance T cell proliferation and IL-2 production in cultures of gastric epithelial cells exposed to H. pylori and CD4 T cells (32), suggesting that PD-L1 blockade may provide a means to improve T cell responses and pathogen control during H. pylori infection. Some examples of other pathogenic bacteria causing infections treatable by methods of the invention include chlamydia, rickettsial bacteria, mycobacteria, staphylococci, streptococci, pneumonococci, meningococci and gonococci, klebsiella, proteus, serratia, pseudomonas, legionella, diphtheria, salmonella, cholera, tetanus, botulism, anthrax, plague, leptospirosis, and Lyme disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Pneumocystis carinii, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Plasmodium vivax, Babesia microti, Trypanosoma brucei, Trypanosoma cruzi, Leishmania donovani, Toxoplasma gondi, and Nippostrongylus brasiliensis.

In all of the above methods, PD-1 modulation can be combined with other forms of immunotherapy such as cytokine treatment (e.g., interferons, GM-CSF, G-CSF, IL-2), or bispecific antibody therapy, which provides for enhanced presentation of tumor antigens.

### Autoimmune Reactions

Compounds of the invention, which are PD-1/PD-L1 agonists, may be used to treat autoimmune diseases, as studies in disease models indicate that the PD-1:PDL pathway is involved in a number of different autoimmune diseases. For example, the PD-1 receptor has been demonstrated to play a role in the development of diabetes. Using a nonobese diabetic (NOD) mouse model it has been demonstrated that blockade of PD-1 receptor, or PD-L1, but not PD-L2 ligands accelerates onset of diabetes. There are a higher percentage of mice developing diabetes than in the control animals. This may be due to the role of PD-1:PDL1 interactions in limiting T cell mediation of autoimmune B cell destruction or controlling the balance between pathogenic effector T cells and protective regulatory T cells. Several studies suggest that PD-1:PD-L1 may mediate apoptosis of activated T cells. These data could provide the rationale for potential therapeutic use of agonists of the PD-1:PD-L pathway in the treatment of autoimmune diseases, such as diabetes (45).

PD-L1 is expressed in parenchymal cells, and non-hematopoietic expression of PD-L1 was demonstrated to be able to protect against autoimmune diabetes using bone marrow chimeras where mice lacking both PD-L1 and PD-L2 were compared with mice lacking either PD-L1 or PD-L2. This study shows PD-L1/PD-L2 expression on APCs is insufficient to prevent early onset diabetes that develops in PDL1/PDL2 knockout mice. Therefore, PD-L1 expression on tissues is a critical mediator of peripheral T cell tolerance. This finding supports the idea that the exploitation of the PD1:PD-L pathway by therapeutic intervention can be used to treat autoimmune disease (40).

The PD-1:PD-L1 pathway also has a role in the regulation of experimental autoimmune encephalomyelitis (EAE). EAE mouse models are used to investigate the autoimmune disease, multiple sclerosis. In this study myelin oligodendrocyte glycoprotein (MOG) was used to induce EAE in mice. MOG induces an immune response that attacks cells of the central nervous system of the mouse. It is reported that after immunization with MOG, expression of PD-1 and PD-L1, but not PD-L2, increased in cells of the central nervous system. Also blockade of the PD-1 receptor by antibodies, leads to accelerated and more severe EAE disease progression. It has been suggested that this result supports the idea that the PD-1:PD-L pathway regulates peripheral tolerance. This result also supports the idea that this pathway could be important in the treatment of many autoimmune diseases (45). The therapeutic potential of this pathway in autoimmune disease is illustrated by a study in which ES cell derived-DCs engineered to have high expression of PD-L1 and myelin antigen could ameliorate EAE and diminish infiltration of the spinal cord by macrophages and T cells (47).

Induction of anti-tumor responses using tumor cell and peptide vaccines reveals that many anti-tumor responses involve anti-self reactivities (depigmentation observed in anti-CTLA-4+GM-CSF-modified B 16 melanoma in van Elsas et al. supra; depigmentation in Trp-2 vaccinated mice; autoimmune prostatitis evoked by TRAMP tumor cell vaccines; and melanoma peptide antigen vaccination and vitilago observed in human clinical trials.

Therefore, it is possible to consider using compounds of the invention in conjunction with various self proteins in order to devise vaccination protocols to efficiently generate immune responses against these self proteins for disease treatment. For example, Alzheimers disease involves inappropriate accumulation of Aβ peptide in amyloid deposits in the brain; antibody responses against amyloid are able to clear these amyloid deposits.

Other self proteins may also be used as targets such as IgE for the treatment of allergy and asthma, and TNFα for rheumatoid arthritis. Finally, antibody responses to various hormones may be induced by the use of a compound of the invention. Neutralizing antibody responses to reproductive hormones may be used for contraception. Neutralizing antibody response to hormones and other soluble factors that are required for the growth of particular tumors may also be considered as possible vaccination targets.

Analogous methods as described above for the use of compounds of the invention can be used for induction of therapeutic autoimmune responses to treat patients having an inappropriate accumulation of other self-antigens, such as amyloid deposits, including Aβ in Alzheimer's disease, cytokines such as TNFα, and IgE.

Additionally, PD-1 has been found to play a role in graft-versus-host disease (Blazar et al J Immuno 2003, 171:1272-7). For example, bone marrow transplantation is currently being used to treat a variety of tumors of hematopoietic origin. While graft versus host disease is a consequence of this treatment, therapeutic benefit may be obtained from graft vs. tumor responses. PD-1 modulation can be used to increase the effectiveness of the donor engrafted tumor specific T cells.

PD-1 also regulates alloimmune responses and graft rejection following solid organ transplantation (Yang et al. Circulation 2008; 117:660-9). For example, PD-1 blockade leads to rapid expansion of graft reactive T cells and graft loss (Koehn et al J Immunol 2008; 181: 5313-22).

It follows that one aspect of the invention related to a method for suppressing, treating, or preventing graft rejection accompanying the transplantation of an organ, or a portion thereof, or a tissue in a subject, the method comprising administering a therapeutically effective amount of a compound or pharmaceutical composition of the invention to the subject. In certain embodiments, the present invention relates to any one of the aforementioned methods, wherein the transplantation is allotransplantation. In certain embodiments, the present invention relates to any one of the aforementioned methods, wherein the transplantation is xenotransplantation. In certain embodiments, the present invention relates to any one of the aforementioned methods, wherein the organ is the liver, heart, kidney, lung, pancreas. In certain embodiments, the present invention relates to any one of the aforementioned methods, wherein the tissue is the skin, cornea, or bone tissue.

Another aspect of the invention relates to a method for enhancing the effect of at least one immunosuppressive agent on the suppression, treatment, or prevention of graft rejection accompanying the transplantation of an organ, or a portion thereof, or a tissue in a subject, the method comprising administering a therapeutically effective amount of a compound or pharmaceutical composition of the invention to the subject. In certain embodiments, the present invention relates to any one of the aforementioned methods, wherein the at least one immunosuppressive agent is selected from the group consisting of azathioprine, adrenocortical steroid, mizoribine, mycophenolate mofetil, leflunomide, sirolimus, deoxyspergualin, FTY720, abatacept, belatacept, an anti-CTLA antibody, cyclosporin, and tacrolimus (FK-506).

### Vaccines

Compounds of the invention may be used to stimulate antigen-specific immune responses by coadministration of a compound of the invention with an antigen of interest (e.g., a vaccine). Accordingly, in another aspect the invention provides a method of enhancing an immune response to an antigen in a subject, comprising administering to the subject: (i) the antigen; and (ii) a compound of the invention, such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

Co-Administration with Other Therapeutic Agents

As previously described, compounds of the invention can be co-administered with one or more other therapeutic agents, e.g., a cytotoxic agent, a radiotoxic agent or an immunosuppressive agent. The compound can be linked to the agent or can be administered separate from the agent. In the latter case (separate administration), the compound can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin, bleomycin sulfate, carmustine, chlorambucil, decarbazine and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the compound of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

### DEFINITIONS

For convenience, certain terms employed in the specification, examples, and appended claims are collected here. All definitions, as defined and used herein, supersede dictionary definitions, definitions in documents incorporated by reference, and/or ordinary meanings of the defined terms.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “solvate” refers to a pharmaceutically acceptable form of a specified compound, with one or more solvent molecules, that retains the biological effectiveness of such compound. Examples of solvates include compounds of the invention in combination with solvents such, for example, water (to form the hydrate), isopropanol, ethanol, methanol, dimethyl sulfoxide, ethyl acetate, acetic acid, ethanolamine, or acetone. Also included are formulations of solvate mixtures such as a compound of the invention in combination with two or more solvents.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “lower” when appended to any of the groups listed below indicates that the group contains less than seven carbons (i.e., six carbons or less). For example “lower alkyl” refers to an alkyl group containing 1-6 carbons, and “lower alkenyl” refers to an alkyenyl group containing 2-6 carbons.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., spiro, fused, bridged).

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

The term “hydrocarbon” as used herein refers to an organic compound consisting entirely of hydrogen and carbon.

For purposes of this invention, the chemical elements are identified in accordance with the Periodic Table of the Elements, CAS version, Handbook of Chemistry and Physics, 67th Ed., 1986-87, inside cover.

The term “heteroatom” as used herein is art-recognized and refers to an atom of any element other than carbon or hydrogen. Illustrative heteroatoms include boron, nitrogen, oxygen, phosphorus, sulfur and selenium.

The term “alkyl” means an aliphatic or cyclic hydrocarbon radical containing from 1 to 20, 1 to 15, or 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl.

The term “alkenyl” as used herein means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl” as used herein means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene,” is art-recognized, and as used herein pertains to a bidentate moiety obtained by removing two hydrogen atoms of an alkyl group, as defined above.

The term “carbocyclyl” as used herein means monocyclic or multicyclic (e.g., bicyclic, tricyclic, etc.) hydrocarbons containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “heterocyclyl”, as used herein include non-aromatic, ring systems, including, but not limited to, monocyclic, bicyclic and tricyclic rings, which can be completely saturated or which can contain one or more units of unsaturation, for the avoidance of doubt, the degree of unsaturation does not result in an aromatic ring system, and have 3 to 12 atoms including at least one heteroatom, such as nitrogen, oxygen, or sulfur. For purposes of exemplification, which should not be construed as limiting the scope of this invention, the following are examples of heterocyclic rings: azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkyenyloxy, alkynyloxy, carbocyclyloxy, heterocyclyloxy, haloalkoxy, fluoroalkyloxy, sulfhydryl, alkylthio, haloalkylthio, fluoroalkylthio, alkyenylthio, alkynylthio, sulfonic acid, alkylsulfonyl, haloalkylsulfonyl, fluororalkylsulfonyl, alkenylsulfonyl, alkynylsulfonyl, alkoxysulfonyl, haloalkoxysulfonyl, fluororalkoxysulfonyl, alkenyloxysulfonyl, alkynyloxysulfony, aminosulfonyl, sulfinic acid, alkylsulfinyl, haloalkylsulfinyl, fluororalkylsulfinyl, alkenylsulfinyl, alkynylsulfinyl, alkoxysulfinyl, haloalkoxysulfinyl, fluororalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluororalkylsulfonyloxy, alkenylsulfonyloxy, alkynylsulfonyloxy, haloalkoxysulfonyloxy, fluororalkoxysulfonyloxy, alkenyloxysulfonyloxy, alkynyloxysulfonyloxy, alkylsulfinyloxy, haloalkylsulfinyloxy, fluororalkylsulfinyloxy, alkenylsulfinyloxy, alkynylsulfinyloxy, alkoxysulfinyloxy, haloalkoxysulfinyloxy, fluororalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g., methylene).

The term “aryl,” as used herein means a phenyl group, naphthyl, phenanthrenyl, or anthracenyl group. The aryl groups of the present invention can be optionally substituted with 1, 2, 3, 4 or 5 substituents independently selected from the group consisting of alkyl, alkenyl, alkynyl, halo, haloalkyl, fluoroalkyl, hydroxy, alkoxy, alkyenyloxy, alkynyloxy, carbocyclyloxy, heterocyclyloxy, haloalkoxy, fluoroalkyloxy, sulfhydryl, alkylthio, haloalkylthio, fluoroalkylthio, alkyenylthio, alkynylthio, sulfonic acid, alkylsulfonyl, haloalkylsulfonyl, fluororalkylsulfonyl, alkenylsulfonyl, alkynylsulfonyl, alkoxysulfonyl, haloalkoxysulfonyl, fluororalkoxysulfonyl, alkenyloxysulfonyl, alkynyloxysulfony, aminosulfonyl, sulfinic acid, alkylsulfinyl, haloalkylsulfinyl, fluororalkylsulfinyl, alkenylsulfinyl, alkynylsulfinyl, alkoxysulfinyl, haloalkoxysulfinyl, fluororalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluororalkylsulfonyloxy, alkenylsulfonyloxy, alkynylsulfonyloxy, haloalkoxysulfonyloxy, fluororalkoxysulfonyloxy, alkenyloxysulfonyloxy, alkynyloxysulfonyloxy, alkylsulfinyloxy, haloalkylsulfinyloxy, fluororalkylsulfinyloxy, alkenylsulfinyloxy, alkynylsulfinyloxy, alkoxysulfinyloxy, haloalkoxysulfinyloxy, fluororalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heterocyclyl group through an alkylene moiety (e.g., methylene).

The term “arylene,” is art-recognized, and as used herein pertains to a bidentate moiety obtained by removing two hydrogen atoms of an aryl ring, as defined above.

The term “arylalkyl” or “aralkyl” as used herein means an aryl group, as defined herein, appended to the parent molecular moiety through an alkyl group, as defined herein. Representative examples of aralkyl include, but are not limited to, benzyl, 2-phenylethyl, 3-phenylpropyl, and 2-naphth-2-ylethyl.

The term “heteroaryl” as used herein include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention are substituted with 0, 1, 2, 3, 4 or 5 substituents independently selected from the group consisting of alkyl, alkenyl, alkynyl, halo, haloalkyl, fluoroalkyl, hydroxy, alkoxy, alkyenyloxy, alkynyloxy, carbocyclyloxy, heterocyclyloxy, haloalkoxy, fluoroalkyloxy, sulfhydryl, alkylthio, haloalkylthio, fluoroalkylthio, alkyenylthio, alkynylthio, sulfonic acid, alkylsulfonyl, haloalkylsulfonyl, fluororalkylsulfonyl, alkenylsulfonyl, alkynylsulfonyl, alkoxysulfonyl, haloalkoxysulfonyl, fluororalkoxysulfonyl, alkenyloxysulfonyl, alkynyloxysulfony, aminosulfonyl, sulfinic acid, alkylsulfinyl, haloalkylsulfinyl, fluororalkylsulfinyl, alkenylsulfinyl, alkynylsulfinyl, alkoxysulfinyl, haloalkoxysulfinyl, fluororalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxy, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluororalkylsulfonyloxy, alkenylsulfonyloxy, alkynylsulfonyloxy, haloalkoxysulfonyloxy, fluororalkoxysulfonyloxy, alkenyloxysulfonyloxy, alkynyloxysulfonyloxy, alkylsulfinyloxy, haloalkylsulfinyloxy, fluororalkylsulfinyloxy, alkenylsulfinyloxy, alkynylsulfinyloxy, alkoxysulfinyloxy, haloalkoxysulfinyloxy, fluororalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, silyloxy, and any of said substituents bound to the heteroaryl group through an alkylene moiety (e.g., methylene).

The term “heteroarylene,” is art-recognized, and as used herein pertains to a bidentate moiety obtained by removing two hydrogen atoms of a heteroaryl ring, as defined above.

The term “heteroarylalkyl” or “heteroaralkyl” as used herein means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “biaryl,” as used herein means an aryl-substituted aryl, an aryl-substituted heteroaryl, a heteroaryl-substituted aryl or a heteroaryl-substituted heteroaryl, wherein aryl and heteroaryl are as defined herein. Representative examples include 4-(phenyl)phenyl and 4-(4-methoxyphenyl)pyridinyl.

The term “halo” or “halogen” means —Cl, —Br, —I or —F.

The term “haloalkyl” means an alkyl group, as defined herein, wherein at least one hydrogen is replaced with a halogen, as defined herein. Representative examples of haloalkyl include, but are not limited to, chloromethyl, 2-fluoroethyl, trifluoromethyl, pentafluoroethyl, and 2-chloro-3-fluoropentyl.

The term “fluoroalkyl” means an alkyl group, as defined herein, wherein some or all of the hydrogens are replaced with fluorines.

The term “hydroxy” as used herein means an —OH group.

The term “alkoxy” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkyenyloxy”, “alkynyloxy”, “carbocyclyloxy”, and “heterocyclyloxy” are likewise defined.

The term “haloalkoxy” as used herein means an alkoxy group, as defined herein, wherein at least one hydrogen is replaced with a halogen, as defined herein. Representative examples of haloalkoxy include, but are not limited to, chloromethoxy, 2-fluoroethoxy, trifluoromethoxy, and pentafluoroethoxy. The term “fluoroalkyloxy” is likewise defined.

The term “aryloxy” as used herein means an aryl group, as defined herein, appended to the parent molecular moiety through an oxygen. The term “heteroaryloxy” as used herein means a heteroaryl group, as defined herein, appended to the parent molecular moiety through an oxygen. The terms “heteroaryloxy” is likewise defined.

The term “arylalkoxy” or “arylalkyloxy” as used herein means an arylalkyl group, as defined herein, appended to the parent molecular moiety through an oxygen. The term “heteroarylalkoxy” is likewise defined. Representative examples of aryloxy and heteroarylalkoxy include, but are not limited to, 2-chlorophenylmethoxy, 3-trifluoromethyl-phenylethoxy, and 2,3-dimethylpyridinylmethoxy.

The term “sulfhydryl” or “thio” as used herein means a —SH group.

The term “alkylthio” as used herein means an alkyl group, as defined herein, appended to the parent molecular moiety through a sulfur. Representative examples of alkylthio include, but are not limited, methylthio, ethylthio, tert-butylthio, and hexylthio. The terms “haloalkylthio”, “fluoroalkylthio”, “alkyenylthio”, “alkynylthio”, “carbocyclylthio”, and “heterocyclylthio” are likewise defined.

The term “arylthio” as used herein means an aryl group, as defined herein, appended to the parent molecular moiety through a sulfur. The term “heteroarylthio” is likewise defined.

The term “arylalkylthio” or “aralkylthio” as used herein means an arylalkyl group, as defined herein, appended to the parent molecular moiety through a sulfur. The term “heteroarylalkylthio” is likewise defined.

The term “sulfonyl” as used herein refers to —S(═O)₂— group.

The term “sulfonic acid” as used herein refers to —S(═O)₂OH.

The term “alkylsulfonyl” as used herein means an alkyl group, as defined herein, appended to the parent molecular moiety through a sulfonyl group, as defined herein. Representative examples of alkylsulfonyl include, but are not limited to, methylsulfonyl and ethylsulfonyl. The terms “haloalkylsulfonyl”, “fluororalkylsulfonyl”, “alkenylsulfonyl”, “alkynylsulfonyl”, “carbocyclylsulfonyl”, “heterocyclylsulfonyl”, “arylsulfonyl”, “aralkylsulfonyl”, “heteroarylsulfonyl” and “heteroaralkylsulfonyl” are likewise defined.

The term “alkoxysulfonyl” as used herein means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl. The terms “haloalkoxysulfonyl”, “fluororalkoxysulfonyl”, “alkenyloxysulfonyl”, “alkynyloxysulfonyl”, “carbocyclyloxysulfonyl”, “heterocyclyloxysulfonyl”, “aryloxysulfonyl”, “aralkyloxysulfonyl”, “heteroaryloxysulfonyl” and “heteroaralkyloxysulfonyl” are likewise defined.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The term “aminosulfonyl” as used herein means an amino group, as defined herein, appended to the parent molecular moiety through a sulfonyl group.

The term “sulfinyl” as used herein refers to —S(═O)— group. Sulfinyl groups are as defined above for sulfonyl groups. The term “sulfinic acid” as used herein refers to —S(═O)OH.

The term “oxy” refers to a —O— group.

The term “carbonyl” as used herein means a —C(═O)— group.

The term “thiocarbonyl” as used herein means a —C(═S)— group.

The term “formyl” as used herein means a —C(═O)H group.

The term “alkylcarbonyl” as used herein means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl. The terms “haloalkylcarbonyl”, “fluoroalkylcarbonyl”, “alkenylcarbonyl”, “alkynylcarbonyl”, “carbocyclylcarbonyl”, “heterocyclylcarbonyl”, “arylcarbonyl”, “aralkylcarbonyl”, “heteroarylcarbonyl”, and “heteroaralkylcarbonyl” are likewise defined.

The term “carboxy” as used herein means a —CO₂H group.

The term “alkoxycarbonyl” as used herein means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl. The terms “haloalkoxycarbonyl”, “fluoroalkoxycarbonyl”, “alkenyloxycarbonyl”, “alkynyloxycarbonyl”, “carbocyclyloxycarbonyl”, “heterocyclyloxycarbonyl”, “aryloxycarbonyl”, “aralkyloxycarbonyl”, “heteroaryloxycarbonyl”, and “heteroaralkyloxycarbonyl” are likewise defined.

The term “alkylcarbonyloxy” as used herein means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. The terms “haloalkylcarbonyloxy”, “fluoroalkylcarbonyloxy”, “alkenylcarbonyloxy”, “alkynylcarbonyloxy”, “carbocyclylcarbonyloxy”, “heterocyclylcarbonyloxy”, “arylcarbonyloxy”, “aralkylcarbonyloxy”, “heteroarylcarbonyloxy”, and “heteroaralkylcarbonyloxy” are likewise defined.

The term “alkylsulfonyloxy” as used herein means an alkylsulfonyl group, as defined herein, appended to the parent molecular moiety through an oxygen atom. The terms “haloalkylsulfonyloxy”, “fluororalkylsulfonyloxy”, “alkenylsulfonyloxy”, “alkynylsulfonyloxy”, “carbocyclylsulfonyloxy”, “heterocyclylsulfonyloxy”, “arylsulfonyloxy”, “aralkylsulfonyloxy”, “heteroarylsulfonyloxy”, “heteroaralkylsulfonyloxy”, “haloalkoxysulfonyloxy”, “fluororalkoxysulfonyloxy”, “alkenyloxysulfonyloxy”, “alkynyloxysulfonyloxy”, “carbocyclyloxysulfonyloxy”, “heterocyclyloxysulfonyloxy”, “aryloxysulfonyloxy”, “aralkyloxysulfonyloxy”, “heteroaryloxysulfonyloxy” and “heteroaralkyloxysulfonyloxy” are likewise defined.

The term “amino” as used herein refers to —NH₂ and substituted derivatives thereof wherein one or both of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkylcarbonyl, haloalkylcarbonyl, fluoroalkylcarbonyl, alkenylcarbonyl, alkynylcarbonyl, carbocyclylcarbonyl, heterocyclylcarbonyl, arylcarbonyl, aralkylcarbonyl, heteroarylcarbonyl, heteroaralkylcarbonyl and the sulfonyl and sulfinyl groups defined above; or when both hydrogens together are replaced with an alkylene group (to form a ring which contains the nitrogen). Representative examples include, but are not limited to methylamino, acetylamino, and dimethylamino.

The term “amido” as used herein means an amino group, as defined herein, appended to the parent molecular moiety through a carbonyl.

The term “cyano” as used herein means a —C≡N group.

The term “nitro” as used herein means a —NO₂ group.

The term “azido” as used herein means a —N₃ group.

The term “phosphinyl” or “phosphino” as used herein includes —PH₃ and substituted derivatives thereof wherein one, two or three of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkoxy, haloalkoxy, fluoroalkyloxy, alkenyloxy, alkynyloxy, carbocyclyloxy, heterocyclyloxy, aryloxy, aralkyloxy, heteroaryloxy, heteroaralkyloxy, and amino.

The term “phosphoryl” as used herein refers to —P(═O)OH₂ and substituted derivatives thereof wherein one or both of the hydroxyls are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkoxy, haloalkoxy, fluoroalkyloxy, alkenyloxy, alkynyloxy, carbocyclyloxy, heterocyclyloxy, aryloxy, aralkyloxy, heteroaryloxy, heteroaralkyloxy, and amino.

The term “silyl” as used herein includes H₃Si— and substituted derivatives thereof wherein one, two or three of the hydrogens are independently replaced with substitutuents selected from alkyl, haloalkyl, fluoroalkyl, alkenyl, alkynyl, carbocyclyl, heterocyclyl, aryl, aralkyl, heteroaryl, and heteroaralkyl. Representative examples include trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, is appended to the parent molecule through an oxygen atom.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The term “treating” as used herein, encompasses the administration and/or application of one or more compounds described herein, to a subject, for the purpose of providing prevention of or management of, and/or remedy for a condition. “Treatment” for the purposes of this disclosure, may, but does not have to, provide a cure; rather, “treatment” may be in the form of management of the condition. When the compounds described herein are used to treat unwanted proliferating cells, including cancers, “treatment” includes partial or total destruction of the undesirable proliferating cells with minimal destructive effects on normal cells. A desired mechanism of treatment of unwanted rapidly proliferating cells, including cancer cells, at the cellular level is apoptosis.

The term “preventing” as used herein includes either preventing or slowing the onset of a clinically evident disease progression altogether or preventing or slowing the onset of a preclinically evident stage of a disease in individuals at risk. This includes prophylactic treatment of those at risk of developing a disease.

The term “subject” for purposes of treatment includes any human or animal subject who has been diagnosed with, has symptoms of, or is at risk of developing a disorder. For methods of prevention the subject is any human or animal subject. To illustrate, for purposes of prevention, a subject may be a human subject who is at risk of or is genetically predisposed to obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds described herein are also useful for veterinary treatment of mammals, including companion animals and farm animals, such as, but not limited to dogs, cats, horses, cows, sheep, and pigs.

The term “modulate” (and grammatical equivalents) refers to an increase or decrease in activity. In particular embodiments, the term “increase” or “enhance” (and grammatical equivalents) means an elevation by at least about 25%, 50%, 75%, 2-fold, 3-fold, 5-fold, 10-fold, 15-fold, 20-fold or more. In particular embodiments, the terms “decrease” or “reduce” (and grammatical equivalents) means a diminishment by at least about 25%, 40%, 50%, 60%, 75%, 80%, 85%, 90%, 95%, 98% or more. In some embodiments, the indicated activity, substance or other parameter is not detectable.

The term “immune response” refers to the action of, for example, lymphocytes, antigen presenting cells, phagocytic cells, granulocytes, and soluble macromolecules produced by the above cells, the liver or mucosal barriers (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, benign prostatic hypertrophy).

As used herein, “about” or “comprising essentially of” mean within an acceptable error range for the particular value as determined by one of ordinary skill in the art, which will depend in part on how the value is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

### EXEMPLIFICATION

The invention now being generally described, it will be more readily understood by reference to the following examples, which are included merely for purposes of illustration of certain aspects and embodiments of the present invention, and are not intended to limit the invention.

Chemical names used below were generated using ChemDraw Ultra version 11.0.

### Preparation of N-(5-methyl-1,3,4-thiadiazol-2-yl)-4-(methylamino)benzenesulfonamide

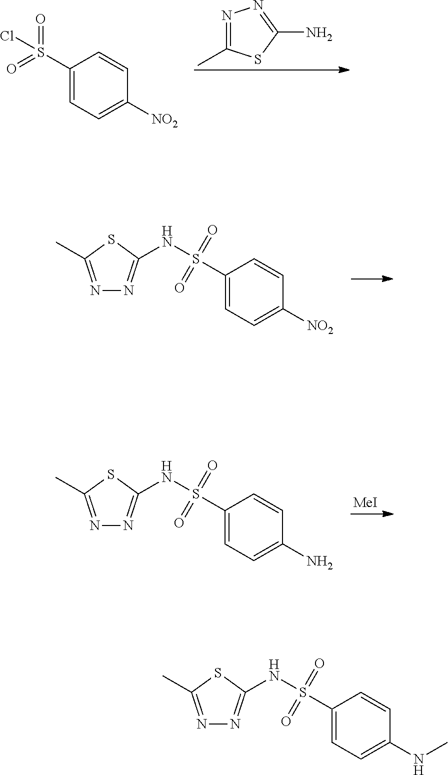

To a solution of 5-methyl-1,3,4-thiadiazol-2-amine (20 g, 5.8 mmol) in pyridine (19 mL, 11.6 mmol) and DCM (100 mL) was added 4-nitrobenzene-1-sulfonyl chloride (25.6 g, 8.69 mmol) in DCM (50 mL). The mixture was stirred at RT for two hours. The solvent was removed under reduced pressure to give a solid (9 g). The solid was purified by column chromatography to give N-(5-methyl-1,3,4-thiadiazol-2-yl)-4-nitrobenzenesulfonamide. MS (ESI) m/z 301 [M+H]⁺.

To a solution of N-(5-methyl-1,3,4-thiadiazol-2-yl)-4-nitrobenzenesulfonamide (300 mg, 1 mmol) in EtOH (8.6 mL) was added Fe (336 mg, 6 mmol) followed by conc. HCl (2 drops) and H₂O (4.3 mL). The reaction was heated to reflux for three hours. Fe was removed by filtration. The filtrate was evaporated to give a solid. The solid was purified by column chromatography to give 4-amino-N-(5-methyl-1,3,4-thiadiazol-2-yl)benzenesulfonamide (300 mg, 100%). ¹H NMR (300 Hz, DMSO-d₆) δ ppm 13.61 (s, 1H), 7.41 (d, 2H), 6.56 (d, 2H), 5.89 (s, 1H), 2.43 (s, 3H). MS (ESI) m/z 271 [M+H]⁺.

To a solution of 4-amino-N-(5-methyl-1,3,4-thiadiazol-2-yl)benzenesulfonamide (200 mg, 0.74 mmol) in DMF (2 mL) was added MeI (0.74 mmol). The mixture was stirred at RT for two days. The solvent was removed under reduced pressure to give a solid. The resulting crude product was purified by column chromatography to give N-(5-methyl-1,3,4-thiadiazol-2-yl)-4-(methylamino)benzenesulfonamide (60 mg, 30%). ¹H NMR (300 Hz, DMSO-d₆) δ ppm 7.42 (d, 2H), 6.48 (d, 2H), 6.21 (s, 1H), 2.66 (d, 3H), 2.36 (s, 3H). MS (ESI) m/z 307.3 [M+Na]⁺.

### Preparation of N-(4-(N-(5-methyl-1,3,4-thiadiazol-2-yl)sulfamoyl)phenyl)acetamide

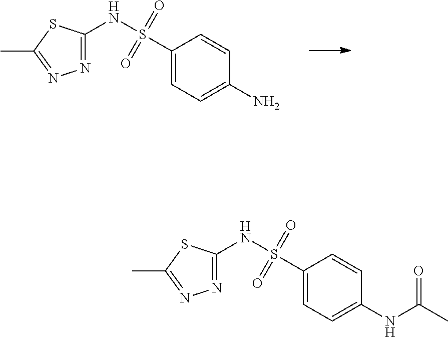 [SMILES: Cc1nnc(NS(=O)(=O)c2ccc(N)cc2)s1 ; CC(=O)Nc1ccc(S(=O)(=O)Nc2nnc(C)s2)cc1]

To a solution of 4-amino-N-(5-methyl-1,3,4-thiadiazol-2-yl)benzenesulfonamide (200 mg, 0.74 mmol) in pyridine (2 mL) was added AcCl (58 mg, 0.74 mmol). The mixture was stirred at RT for two hours. The solvent was removed under reduced pressure to give a solid. The solid was purified by column chromatography to give N-(4-(N-(5-methyl-1,3,4-thiadiazol-2-yl)sulfamoyl)phenyl)acetamide (70 mg, 30.3%). ¹H NMR (300 Hz, DMSO-d₆) δ ppm 13.89 (s, 1H), 10.31 (s, 1H), 7.75 (s, 4H), 2.49 (s, 3H), 2.11 (s, 3H). MS (ESI) m/z 311.0 [M−H]⁻.

### Preparation of Methyl 4-(N-(5-methyl-1,3,4-thiadiazol-2-yl)sulfamoyl)phenylcarbamate

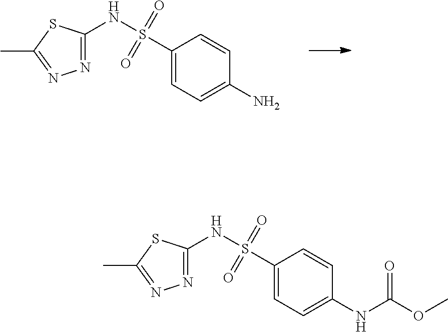

To a solution of 4-amino-N-(5-methyl-1,3,4-thiadiazol-2-yl)benzenesulfonamide (200 mg, 0.74 mmol) in pyridine (2 mL) was added methyl chloroformate (70 mg, 0.74 mmol). The mixture was stirred at RT for two hours. The solvent was removed under reduced pressure to give a solid. The solid was purified by column chromatography to give methyl 4-(N-(5-methyl-1,3,4-thiadiazol-2-yl)sulfamoyl)phenylcarbamate (80 mg, 32.9%). 1H NMR (300 Hz, DMSO-d₆) δ ppm 10.04 (s, 1H), 7.68 (d, 2H), 7.56 (d, 2H), 3.68 (s, 3H), 2.43 (s, 3H). MS (ESI) m/z 351.1 [M+Na]⁺.

### Preparation of N-(5-methyl-1,3,4-thiadiazol-2-yl)-4-(3-methylureido)benzenesulfonamide

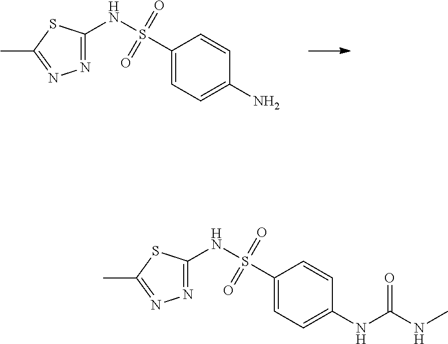

To a solution of 4-amino-N-(5-methyl-1,3,4-thiadiazol-2-yl)benzenesulfonamide (200 mg, 0.74 mmol) in pyridine (2 mL) was added methylcarbamic chloride (69 mg, 0.74 mmol). The mixture was stirred at RT for two hours. The solvent was removed under reduced pressure to give a solid. The solid was purified by column chromatography to give N-(5-methyl-1,3,4-thiadiazol-2-yl)-4-(3-methylureido)benzenesulfonamide (50 mg, 20.6%).

1H NMR (300 Hz, DMSO-d₆) δ ppm 13.89 (s, 1H), 8.94 (s, 1H), 7.62 (d, 2H), 7.53 (d, 2H), 6.14 (s, 1H), 2.64 (s, 3H), 2.45 (s, 3H). MS (ESI) m/z 326.1 [M−H]⁻.

### Preparation of 4-Hydroxy-N-(5-methyl-1,3,4-thiadiazol-2-yl)benzenesulfonamide

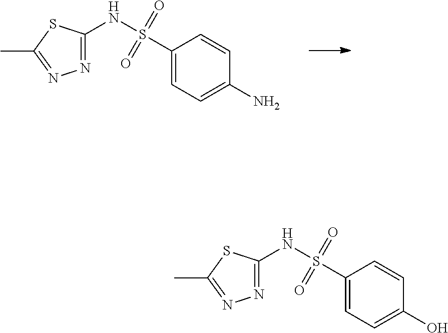 [SMILES: Cc1nnc(NS(=O)(=O)c2ccc(N)cc2)s1 ; Cc1nnc(NS(=O)(=O)c2ccc(O)cc2)s1]

To a solution of conc. H₂SO₄ (0.3 g) in H₂O (1 mL) was added 4-amino-N-(5-methyl-1,3,4-thiadiazol-2-yl)benzenesulfonamide (200 g, 0.74 mmol) followed by a solution of NaNO₂ (51 mg, 0.74 mmol) in water (0.5 mL). The mixture was heated to reflux for five minutes. The solvent was added into chilled water and filtered. Dry the filter cake and give 4-hydroxy-N-(5-methyl-1,3,4-thiadiazol-2-yl)benzenesulfonamide (95 mg, 47.5%). ¹H NMR (300 Hz, DMSO-d₆) δ ppm 13.78 (s, 1H), 10.34 (s, 1H), 7.60 (d, 2H), 6.85 (d, 2H), 2.23 (s, 3H). MS (ESI) m/z 269.8 [M−H]⁻.

### Preparation of 6-Amino-N-(5-methyl-1,3,4-thiadiazol-2-yl)pyridine-3-sulfonamide

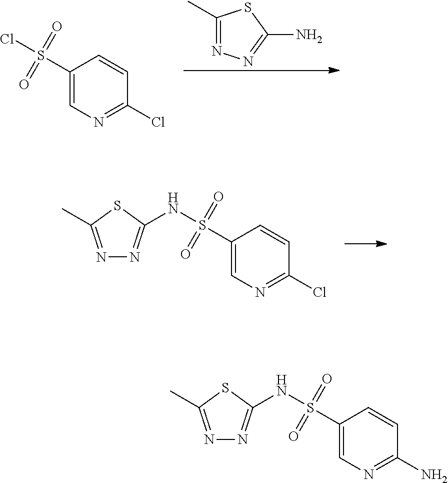

To a solution of 5-methyl-1,3,4-thiadiazol-2-amine (100 mg, 0.799 mmol) in pyridine (5 ml) was added 6-chloropyridine-3-sulfonyl chloride (200 mg, 0.94 mmol). The mixture was stirred at RT for two hours. The solvent was removed under reduced pressure to give a solid. The solid was purified by column chromatography to give 6-chloro-N-(5-methyl-1,3,4-thiadiazol-2-yl)pyridine-3-sulfonamide (25 mg, 10.7%). 1H NMR (300 Hz, DMSO-d6) δ ppm 8.62 (s, 1H), 8.05 (d, 2H), 7.55 (d, 2H), 2.44 (s, 3H). MS (ESI) m/z 288.9 [M−H]⁻.

The mixture of 6-chloro-N-(5-methyl-1,3,4-thiadiazol-2-yl)pyridine-3-sulfonamide (45 mg, 0.155 mmol) in NH₃.H₂O (10 mL) was heated at 110° C. for two days. The solvent was removed under reduced pressure to give a solid. The solid was purified by column chromatography to give amino-N-(5-methyl-1,3,4-thiadiazol-2-yl)pyridine-3-sulfonamide (26 mg, 61.9%). ¹H NMR (300 Hz, DMSO-d₆) δ ppm 8.21 (s, 1H), 7.57 (d, 2H), 6.46 (s, 2H), 6.40 (d, 2H), 2.37 (s, 3H). MS (ESI) m/z 270.0 [M−H]⁻.

### Preparation of 4-Amino-N-(5-methyl-1,3,4-thiadiazol-2-yl)benzamide

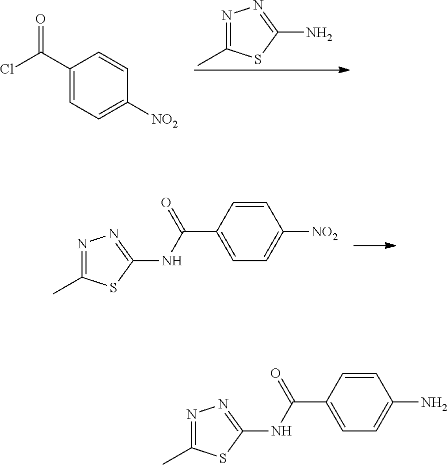

To a solution of 5-methyl-1,3,4-thiadiazol-2-amine (1.0 g, 8.68 mmol) in pyridine (10 mL) was added 4-nitrobenzoyl chloride (3.3 g, 17.37 mmol). The mixture was stirred at RT for two hours. The solvent was removed under reduced pressure to give a solid. The solid was purified by column chromatography to give N-(5-methyl-1,3,4-thiadiazol-2-yl)-4-nitrobenzamide (834 mg, 36.4%). MS (ESI) m/z 263.1 [M−H]⁻

To a solution of N-(5-methyl-1,3,4-thiadiazol-2-yl)-4-nitrobenzamide (478 mg, 1.81 mmol) in EtOH (9.6 ml) was added Fe (608 mg, 10.9 mmol) followed by conc. HCl (3 drops) and H₂O (4.8 mL). The reaction was heated to reflux for three hours. Fe was removed by filtration. The filtrate was evaporated to give a solid. The solid was purified by column chromatography to give 4-amino-N-(5-methyl-1,3,4-thiadiazol-2-yl)benzamide (380 mg, 89.6%). ¹H NMR (300 Hz, DMSO-d₆) δ ppm 11.89 (s, 1H), 7.61 (d, 2H), 6.54 (d, 2H), 5.86 (s, 2H). MS (ESI) m/z 233 [M−H]⁻.

### Preparation of 4-Amino-N-methyl-N-(5-methyl-1,3,4-thiadiazol-2-yl)benzenesulfonamide

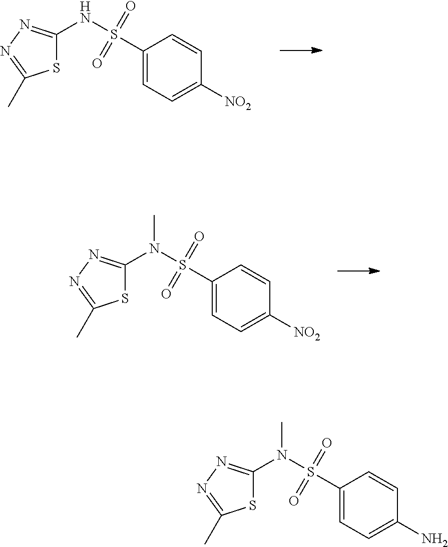 [SMILES: Cc1nnc(NS(=O)(=O)c2ccc([N+](=O)[O-])cc2)s1 ; Cc1nnc(N(C)S(=O)(=O)c2ccc([N+](=O)[O-])cc2)s1 ; Cc1nnc(N(C)S(=O)(=O)c2ccc(N)cc2)s1]

To a solution of NaH (0.1 g, 4.33 mmol) in THF (2 mL) was added a solution of N-(5-methyl-1,3,4-thiadiazol-2-yl)-4-nitrobenzenesulfonamide (1.0 g, 3.33 mmol) in THF (8 mL) followed by MeI (0.95 g, 6.66 mmol). The mixture was stirred at RT for two hours. The mixture was quenched by water. The solvent was removed under reduced pressure to give a solid. The solid was purified by column chromatography to give N-methyl-N-(5-methyl-1,3,4-thiadiazol-2-yl)-4-nitrobenzenesulfonamide (360 mg, 34.6%). MS (ESI) m/z 327.3 [M+Na]⁺.

To a solution of N-methyl-N-(5-methyl-1,3,4-thiadiazol-2-yl)-4-nitrobenzenesulfonamide (200 mg, 0.636 mmol) in EtOH (4 ml) was added Fe (214 mg, 3.82 mmol) followed by conc. HCl (2 drops) and H₂O (2 mL). The reaction was heated to reflux for three hours. Fe was removed by filtration. The filtrate was evaporated to give a solid. The solid was purified by column chromatography to give 4-amino-N-methyl-N-(5-methyl-1,3,4-thiadiazol-2-yl)benzenesulfonamide (178 mg, 98%). ¹H NMR (300 Hz, DMSO-d₆) δ ppm 7.43 (d, 2H), 6.57 (d, 2H), 5.91 (s, 2H), 3.57 (s, 3H), 2.45 (s, 3H). MS (ESI) m/z 307 [M+Na]⁺.

### Preparation of 4-Amino-N-(1,3,4-thiadiazol-2-yl)benzenesulfonamide

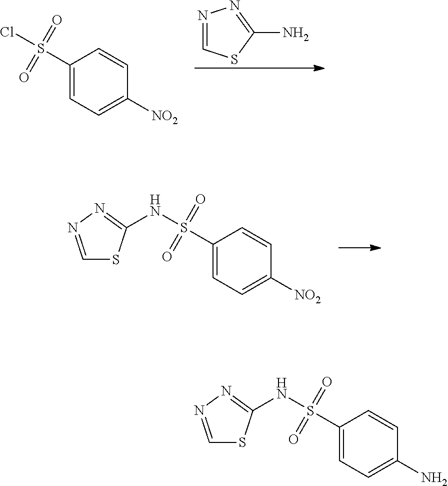 [SMILES: O=[N+]([O-])c1ccc(S(=O)(=O)Cl)cc1 ; Nc1nncs1 ; O=[N+]([O-])c1ccc(S(=O)(=O)Nc2nncs2)cc1 ; Nc1ccc(S(=O)(=O)Nc2nncs2)cc1]

To a solution of 1,3,4-thiadiazol-2-amine1 (5.0 g, 49.44 mmol) in pyridine (130 mL) was added 4-nitrobenzene-1-sulfonyl chloride (7.3 g, 32.96 mmol). The mixture was stirred at RT for two hours. The solvent was removed under reduced pressure to give a solid. The solid was purified by column chromatography to give 4-nitro-N-(1,3,4-thiadiazol-2-yl)benzenesulfonamide (1.82 g, 20%).

To a solution of 4-nitro-N-(1,3,4-thiadiazol-2-yl)benzenesulfonamide (500 mg, 1.7465 mmol) in EtOH (10 mL) was added Fe (587 mg, 10.4789 mmol) followed by conc. HCl (5 drops) and H₂O (5 mL). The reaction was heated to reflux for three hours. Fe was removed by filtration. The filtrate was evaporated to give a solid. The solid was purified by column chromatography to give 4-amino-N-(1,3,4-thiadiazol-2-yl)benzenesulfonamide (138 mg, 31%). ¹H NMR (300 Hz, DMSO-d₆) δ ppm 8.64 (s, 1H), 7.40 (d, 2H), 6.56 (d, 2H), 5.85 (s, 2H). MS (ESI) m/z 254.9 [M−H]⁻.

### Preparation of 4-Amino-N-(5-phenyl-1,3,4-thiadiazol-2-yl)benzenesulfonamide

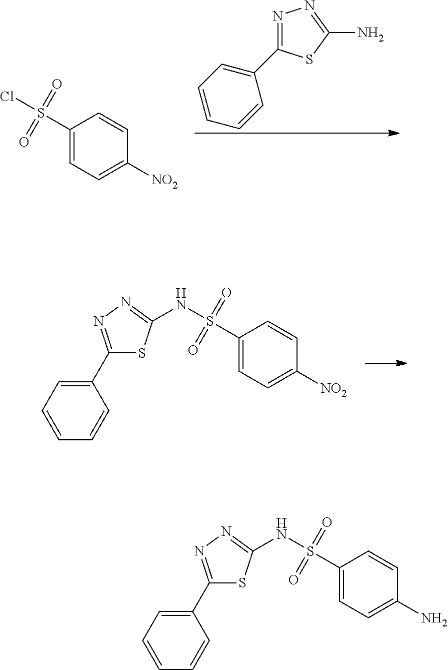 [SMILES: O=[N+]([O-])c1ccc(S(=O)(=O)Cl)cc1 ; Nc1nnc(-c2ccccc2)s1 ; O=[N+]([O-])c1ccc(S(=O)(=O)Nc2nnc(-c3ccccc3)s2)cc1 ; Nc1ccc(S(=O)(=O)Nc2nnc(-c3ccccc3)s2)cc1]

To a solution of 5-phenyl-1,3,4-thiadiazol-2-amine (1.5 g, 8.4 mmol) in pyridine (20 mL) was added 4-nitrobenzene-1-sulfonyl chloride (930 mg, 4.2 mmol) followed by DMAP (69 mg, 0.5462 mmol). The mixture was stirred at RT for two hours. The solvent was removed under reduced pressure to give a solid. The solid was purified by column chromatography to give 4-nitro-N-(5-phenyl-1,3,4-thiadiazol-2-yl)benzenesulfonamide (429 mg, 31%). MS (ESI) m/z 361 [M−H]⁻.

To a solution of 4-nitro-N-(5-phenyl-1,3,4-thiadiazol-2-yl)benzenesulfonamide (340 mg, 0.938 mmol) in EtOH (6.8 mL) was added Fe (315 mg, 5.629 mmol) followed by conc. HCl (2 drops) and H₂O (3.4 mL). The reaction was heated to reflux for three hours. Fe was removed by filtration. The filtrate was evaporated to give a solid. The solid was purified by column chromatography to give 4-amino-N-(5-phenyl-1,3,4-thiadiazol-2-yl)benzenesulfonamide (360 mg, 100%). ¹H NMR (300 Hz, DMSO-d₆) δ ppm 7.80 (s, 2H), 7.53˜7.46 (m, 6H), 6.58 (s, 2H), 5.94 (s, 2H). MS (ESI) m/z 331 [M−H]⁻.

### Preparation of 4-Amino-N-(5-(methoxymethyl)-1,3,4-thiadiazol-2-yl)benzenesulfonamide

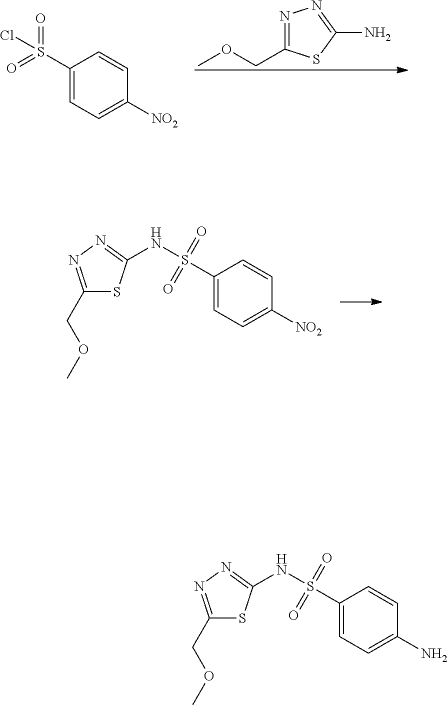

To a solution of 5-(methoxymethyl)-1,3,4-thiadiazol-2-amine (500 mg, 3.444 mmol) in pyridine (5 mL) was added 4-nitrobenzene-1-sulfonyl chloride (1.5 g, 6.888 mmol). The mixture was stirred at RT for two hours. The solvent was removed under reduced pressure to give a solid. The solid was purified by column chromatography to give N-(5-(methoxymethyl)-1,3,4-thiadiazol-2-yl)-4-nitrobenzenesulfonamide (1.0 g, 91%). MS (ESI) m/z 329 [M−H]⁻.

To a solution of N-(5-(methoxymethyl)-1,3,4-thiadiazol-2-yl)-4-nitrobenzenesulfonamide (300 mg, 0.90843 mmol) in EtOH (6 ml) was added Fe (305 mg, 5.45 mmol) followed by conc. HCl (3 drops) and H₂O (3 mL). The reaction was heated to reflux for three hours. Fe was removed by filtration. The filtrate was evaporated to give a solid. The solid was purified by column chromatography to give 4-amino-N-(5-(methoxymethyl)-1,3,4-thiadiazol-2-yl)benzenesulfonamide (273 mg, 100%). ¹H NMR (300 Hz, DMSO-d₆) δ ppm 7.40 (d, 2H), 6.56 (d, 2H), 5.87 (s, 2H), 4.53 (s, 2H), 3.32 (s, 3H). MS (ESI) m/z 301 [M+H]⁺.

### Preparation of 4-Amino-N-(5-methylthiazol-2-yl)benzenesulfonamide

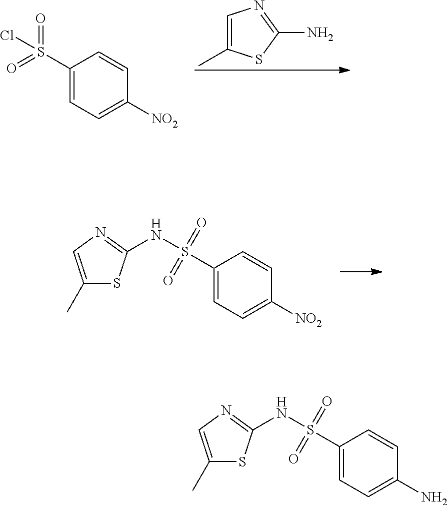

To a solution of 5-methylthiazol-2-amine (500 mg, 4.38 mmol) in pyridine (5 mL) was added 4-nitrobenzene-1-sulfonyl chloride (1.65 g, 5.26 mmol). The mixture was stirred at RT for two hours. The solvent was removed under reduced pressure to give a solid. The solid was purified by column chromatography to give N-(5-methylthiazol-2-yl)-4-nitrobenzenesulfonamide (325 mg, impure).

To a solution of N-(5-methylthiazol-2-yl)-4-nitrobenzenesulfonamide (300 mg, 1.0022 mmol) in EtOH (6 mL) was added Fe (337 mg, 6.01 mmol) followed by conc. HCl (2 drops) and H₂O (3 mL). The reaction was heated to reflux for three hours. Fe was removed by filtration. The filtrate was evaporated to give a solid. The solid was purified by column chromatography to give 4-amino-N-(5-methylthiazol-2-yl)benzenesulfonamide (200 mg, 74%). ¹H NMR (300 Hz, DMSO-d₆) δ ppm 7.40 (d, 1H), 6.85 (s, 0.5H), 6.56 (d, 1H), 5.79 (s, 1H), 2.15 (s, 1.5H). MS (ESI) m/z 268 [M−H]⁻.

### Preparation of 4-Amino-N-(4-methoxypyridin-2-yl)benzenesulfonamide

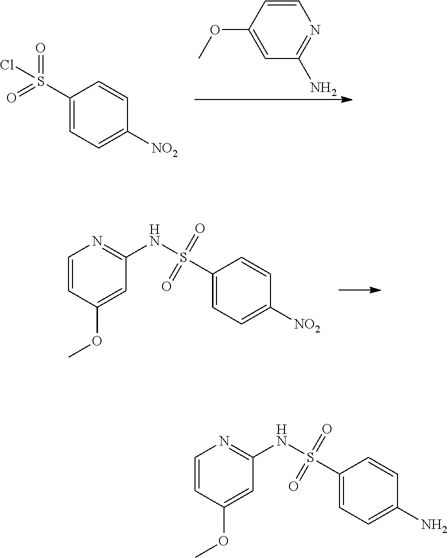

To a solution of 4-methoxypyridin-2-amine (500 mg, 4.03 mmol) in pyridine (5 ml) was added 4-nitrobenzene-1-sulfonyl chloride (1.07 g, 4.83 mmol). The mixture was stirred at RT for two hours. The solvent was removed under reduced pressure to give a solid. The solid was purified by column chromatography to give N-(4-methoxypyridin-2-yl)-4-nitrobenzenesulfonamide (210 mg, %). MS (ESI) m/z 308 [M−H]⁻.

To a solution of N-(4-methoxypyridin-2-yl)-4-nitrobenzenesulfonamide (300 mg, 0.9699 mmol) in EtOH (6 mL) was added Fe (326 mg, 5.819 mmol) followed by conc. HCl (3 drops) and H₂O (3 mL). The reaction was heated to reflux for three hours. Fe was removed by filtration. The filtrate was evaporated to give a solid. The solid was purified by column chromatography to give 4-amino-N-(4-methoxypyridin-2-yl)benzenesulfonamide (180 mg, 67%). ¹H NMR (500 Hz, DMSO-d₆) δ ppm 7.78 (s, 1H), 7.48 (s, 2H), 6.56 (s, 3H), 6.42 (s, 1H), 3.37 (s, 3H). MS (ESI) m/z 280 [M+H]⁺.

### Preparation of 4-Amino-N-(4-methoxypyrimidin-2-yl)benzenesulfonamide

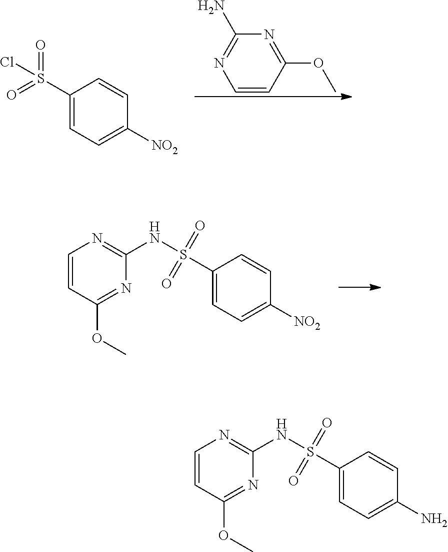

To a solution of 4-methoxypyrimidin-2-amine (600 mg, 4.79 mmol) in pyridine (10 mL) was added 4-nitrobenzene-1-sulfonyl chloride (1.28 g, 5.75 mmol). The mixture was stirred at RT for two hours. The solvent was removed under reduced pressure to give a solid. The solid was purified by column chromatography to give N-(4-methoxypyrimidin-2-yl)-4-nitrobenzenesulfonamide (450 mg, 30%). MS (ESI) m/z 309 [M−H]⁻.

To a solution of N-(4-methoxypyrimidin-2-yl)-4-nitrobenzenesulfonamide (430 mg, 1.3858 mmol) in EtOH (9 ml) was added Fe (465 mg, 8.3148 mmol) followed by conc. HCl (3 drops) and H₂O (4.5 mL). The reaction was heated to reflux for three hours. Fe was removed by filtration. The filtrate was evaporated to give a solid. The solid was purified by column chromatography to give 4-amino-N-(4-methoxypyrimidin-2-yl)benzenesulfonamide (190 mg, 50%). ¹H NMR (300 Hz, DMSO-d₆) δ ppm 8.12 (s, 1H), 7.57 (d, 3H), 6.56 (d, 3H), 6.34 (s, 2H), 3.79 (s, 3H). MS (ESI) m/z 303.0 [M+Na]⁺.

### Preparation of 4-Amino-N-(2-methoxypyridin-4-yl)benzenesulfonamide

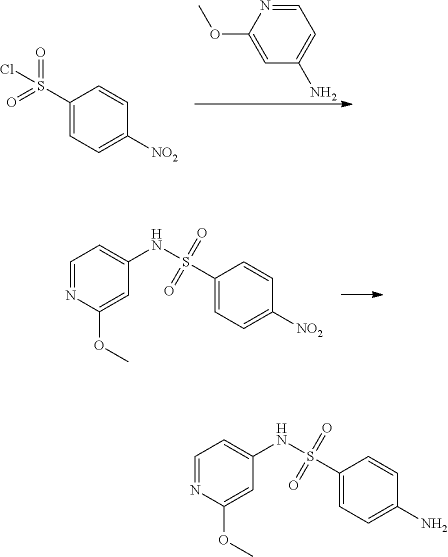

To a solution of 2-methoxypyridin-4-amine (100 mg, 0.8055 mmol) in pyridine (3 mL) was added 4-nitrobenzene-1-sulfonyl chloride (214 mg, 0.9667 mmol). The mixture was stirred at RT for two hours. The solvent was removed under reduced pressure to give a solid. The solid was purified by column chromatography to give N-(2-methoxypyridin-4-yl)-4-nitrobenzenesulfonamide MS (ESI) m/z 308 [M−H]⁻.

To a solution of N-(2-methoxypyridin-4-yl)-4-nitrobenzenesulfonamide (400 mg, 1.2932 mmol) in EtOH (8 mL) was added Fe (435 mg, 7.7595 mmol) followed by conc. HCl (3 drops) and H₂O (4 mL). The reaction was heated to reflux for three hours. Fe was removed by filtration. The filtrate was evaporated to give a solid. The solid was purified by column chromatography to give 4-amino-N-(2-methoxypyridin-4-yl)benzenesulfonamide (100 mg, 23%). ¹H NMR (300 Hz, DMSO-d₆) δ ppm 7.88 (d, 1H), 7.48 (d, 2H), 6.65 (d, 1H), 6.59 (d, 2H), 6.36 (s, 1H), 6.08 (s, 2H), 3.78 (s, 3H). MS (ESI) m/z 280 [M+H]⁺.

### Preparation of 4-Amino-N-(pyrimidin-4-yl)benzenesulfonamide

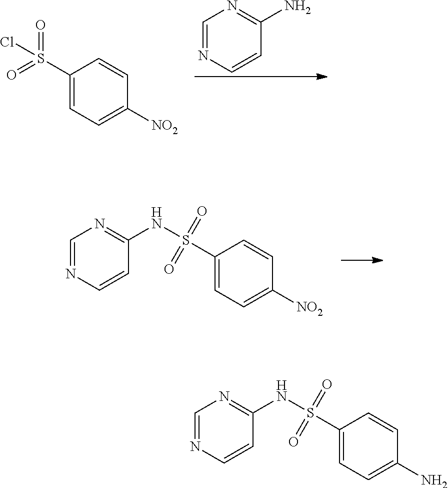 [SMILES: O=[N+]([O-])c1ccc(S(=O)(=O)Cl)cc1 ; Nc1ccncn1 ; O=[N+]([O-])c1ccc(S(=O)(=O)Nc2ccncn2)cc1 ; Nc1ccc(S(=O)(=O)Nc2ccncn2)cc1]

To a solution of pyrimidin-4-amine (200 mg, 2.10305 mmol) in pyridine (4 mL) was added 4-nitrobenzene-1-sulfonyl chloride (923 mg, 4.2061 mmol). The mixture was stirred at RT for two hours. The solvent was removed under reduced pressure to give a solid. The solid was purified by column chromatography to give 4-nitro-N-(pyrimidin-4-yl)benzenesulfonamide (290 mg, 49%). MS (ESI) m/z 279 [M−H]⁻.

To a solution of 4-nitro-N-(pyrimidin-4-yl)benzenesulfonamide (150 mg, 0.5352 mmol) in EtOH (3 ml) was added Fe (180 mg, 3.2113 mmol) followed by conc. HCl (2 drops) and H₂O (1.5 mL). The reaction was heated to reflux for three hours. Fe was removed by filtration. The filtrate was evaporated to give a solid. The solid was purified by column chromatography to give 4-amino-N-(pyrimidin-4-yl)benzenesulfonamide (62 mg). ¹H NMR (300 Hz, DMSO-d₆) δ ppm 8.61 (s, 1H), 8.33 (d, 1H), 7.56 (d, 2H), 6.95 (d, 1H), 6.57 (d, 2H), 6.00 (s, 2H). MS (ESI) m/z 249 [M−H]⁻.

### Preparation of 4-Amino-N-(6-methylpyrimidin-4-yl)benzenesulfonamide

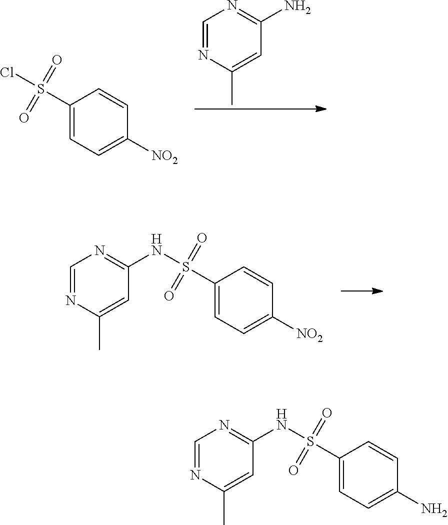

To a solution of 6-methylpyrimidin-4-amine (1.0 g, 4.58 mmol) in pyridine (15 mL) was added 4-nitrobenzene-1-sulfonyl chloride (4 g, 9.16 mmol). The mixture was stirred at RT for two hours. The solvent was removed under reduced pressure to give a solid. The solid was purified by column chromatography to give N-(6-methylpyrimidin-4-yl)-4-nitrobenzenesulfonamide (829 mg, 26%). MS (ESI) m/z 293 [M−H]⁻.

To a solution of N-(6-methylpyrimidin-4-yl)-4-nitrobenzenesulfonamide (300 mg, 1.0194 mmol) in EtOH (6 ml) was added Fe (345 mg, 6.1164 mmol) followed by conc. HCl (3 drops) and H₂O (3 mL). The reaction was heated to reflux for three hours. Fe was removed by filtration. The filtrate was evaporated to give a solid. The solid was purified by column chromatography to give 4-amino-N-(6-methylpyrimidin-4-yl)benzenesulfonamide (60 mg, 22%). 1H NMR (300 Hz, DMSO-d₆) δ ppm 8.48 (s, 1H), 7.56 (d, 2H), 6.81 (s, 1H), 6.56 (d, 2H), 5.99 (s, 2H), 2.29 (s, 3H). MS (ESI) m/z 265 [M+H]⁺.

### Preparation of 4-Amino-N-(6-phenylpyrimidin-4-yl)benzenesulfonamide

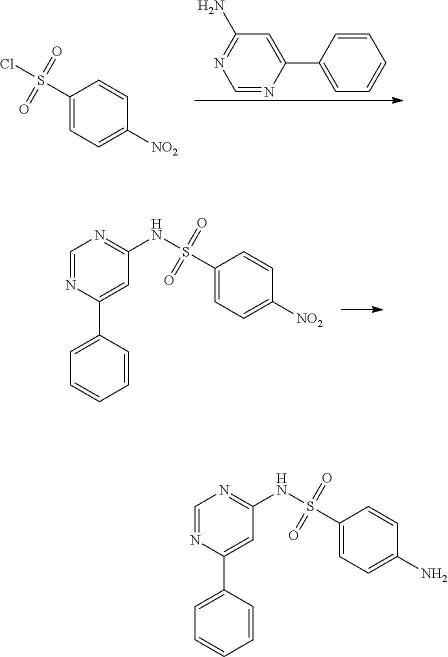 [SMILES: O=[N+]([O-])c1ccc(S(=O)(=O)Cl)cc1 ; Nc1cc(-c2ccccc2)ncn1 ; O=[N+]([O-])c1ccc(S(=O)(=O)Nc2cc(-c3ccccc3)ncn2)cc1 ; Nc1ccc(S(=O)(=O)Nc2cc(-c3ccccc3)ncn2)cc1]

To a solution of 6-phenylpyrimidin-4-amine (170 mg, 0.993 mmol) in pyridine (3 mL) was added 4-nitrobenzene-1-sulfonyl chloride (440 mg, 1.993 mmol). The mixture was stirred at RT for two hours. The solvent was removed under reduced pressure to give a solid. The solid was purified by column chromatography to give 4-nitro-N-(6-phenylpyrimidin-4-yl)benzenesulfonamide (240 mg, 68%). MS (ESI) m/z 354 [M−H]⁻.

To a solution of 4-nitro-N-(6-phenylpyrimidin-4-yl)benzenesulfonamide (240 mg, 0.67348 mmol) in EtOH (4.8 ml) was added Fe (230 mg, 4.04088 mmol) followed by conc. HCl (3 drops) and H₂O (2.4 mL). The reaction was heated to reflux for three hours. Fe was removed by filtration. The filtrate was evaporated to give a solid. The solid was purified by column chromatography to give 4-amino-N-(6-phenylpyrimidin-4-yl)benzenesulfonamide (263 mg, 100%). 1H NMR (300 Hz, DMSO-d₆) δ ppm 11.55 (s, 1H), 8.75 (s, 1H), 7.97 (s, 2H), 7.64 (d, 2H), 7.54 (s, 3H), 7.42 (s, 1H), 6.58 (d, 2H), 6.08 (s, 2H). MS (ESI) m/z 325.3 [M−H]⁻.

### Preparation of 4-Amino-N-(5-methoxypyridin-2-yl)benzenesulfonamide

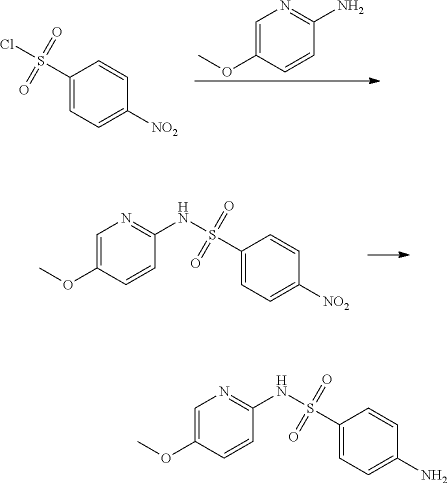

To a solution of 5-methoxypyridin-2-amine (200 mg, 1.6111 mmol) in pyridine (4 mL) was added 4-nitrobenzene-1-sulfonyl chloride (428 mg, 1.9333 mmol). The mixture was stirred at RT for two hours. The solvent was removed under reduced pressure to give a solid (260 mg, 52%). The solid was purified by column chromatography to give N-(5-methoxypyridin-2-yl)-4-nitrobenzenesulfonamide. MS (ESI) m/z 308.3 [M−H]⁻.

### Determining the PD-1 Activity of Sulfonamides

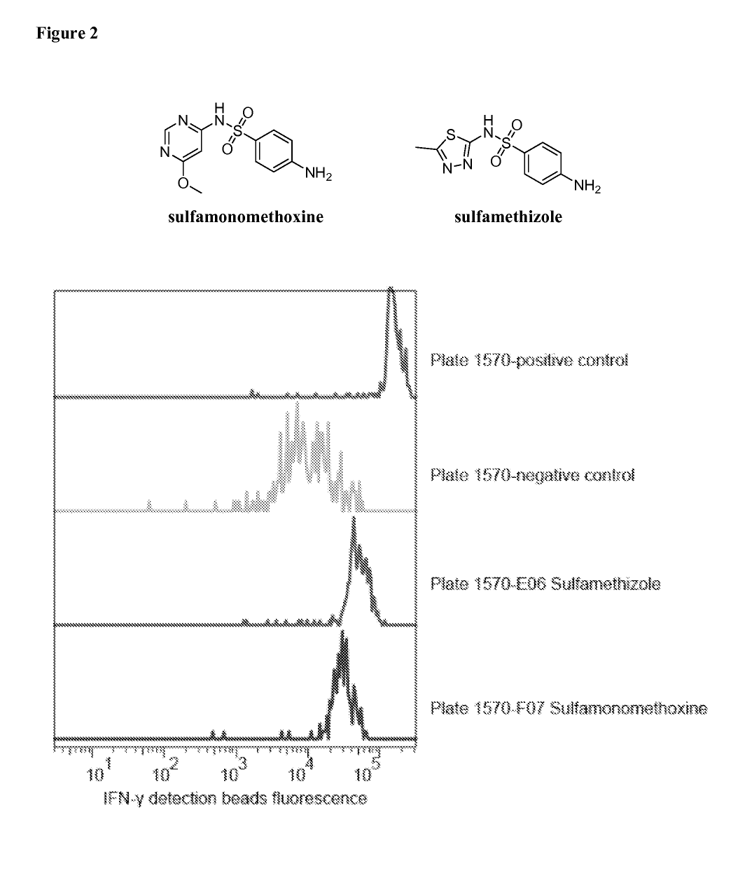

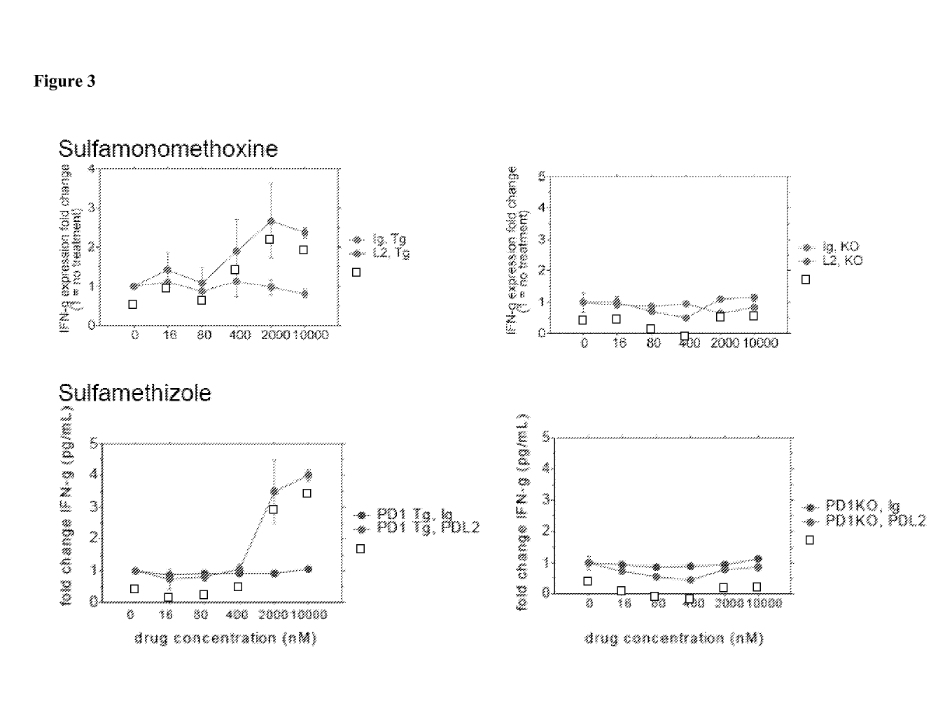

The specificity of the effects of sulfamonomethoxine and sulfamethizole were assessed by comparing responses of wild type, PD-1^{c}, and PD-1^{-/-} T cells. The effects of sulfamonomethoxine and sulfamethizole were evaluated over a range of compound concentrations (dilution series from 16 nM to 80 μM) and analyzed their effects on T cell proliferation and cytokine production. The 2D structure and data from the primary screen are shown in FIG. 2. A secondary screen demonstrated the specificity of sulfamonomethoxine and sulfamethizole by the rescue of PD-1 mediated inhibition in PD-1 Tg cells only when PD-L2 was present. Furthermore, there was no effect on PD-1 KO cells (FIG. 3). Specific effects of sulfamonomethoxine and sulfamethizole are seen above 400 nM in rescuing PD-1 mediated inhibition of IFN-γ production. It can be concluded that both sulfonamides conferred some PD-1-pathway specific rescue.

More detail regarding experimental procedures is provided below.

Obtaining T-Cells.

To obtain T cells the mice were sacrificed. The spleen was harvested from each type of mouse, and processed by crushing the organ through a 10 μM screen filter with the plunger of a syringe. The CD4 T cells were then separated from the rest of the lymphocytes using Miltenyi Biotec LS columns (Cat #130-042-401) with CD4 (L3T4) Microbeads (Cat #130-049-201). The LS columns are composed of ferromagnetic spheres which are coated with a cell friendly plastic coating. The columns have a capacity of up to 1×10⁸ magnetically labeled cells from up to 2×10⁹ total cells. The CD4 (L3T4) Microbeads are antibodies specific for the CD4 receptor conjugated with a magnetic bead. The lymphocytes from the PD^{C} and the PD-1 KO mice are respectively labeled with the CD4 microbeads. The LS column is attached to a strong magnet while the lymphocyte/CD4 microbeads combination is passed through the column. This process allows for positive selection of the CD4 T cells. These cells are then used for the various assays. The table below outlines this procedure.

| | |
|---|---|
| 1 | Obtain PD-1^{C} or PD-1 KO mice and remove spleen |
| 2 | Mash Spleen through 10 μM screen filter |
| | Wash filter w/ serum free media (RPMI) |
| 3 | Spin down cells for 5 minutes @ 1250 rpm |
| | Wash cells 2X with RPMI |
| 4 | Count cells and access viability |
| 5 | Resuspend cells in MACs buffer (PBS w/ 0.5% BSA & 2 mM |
| | EDTA) @ 90 μL/10⁷ cells |
| 6 | Add CD4 (L3T4) Microbeads |
| | @ 7 μL/10⁷ cells |
| | Mix well and incubate for 15 minutes @ 4° C. |
| 7 | After incubation Wash bead/cell mixture by adding 40 mls of |
| | MACs buffer |
| | Spin down cells for 5 minutes @ 1250 rpm |
| | Remove supernatant |
| 8 | Resuspend cell pellet with 1 ml of MACs buffer |
| 9 | Prepare LS column |
| | Place column in Magnetic holder & stand |
| | Equilibrate column with 3 mls of MACs buffer |
| 10 | Add cells to the column |
| 11 | Wash LS columns |
| | Wash 3× with 3 mls of MACs buffer |
| 12 | Remove column from Magnetic holder |
| | Elute column with 5 mls of MACs buffer |
| 13 | Add 5 mls of MACs buffer and Count Cells |
| 14 | Wash cells 2× in RPMI with 10% fetal bovine serum (FBS), |
| | 1M Hepes buffer, 1% Antibiotic, 1% L-Glutamine and 0.1M of |
| | β-mercaptoethanol (RPMI with these components will be |
| | referred to as CR10 media) |
| 15 | Resuspend cells in CR10 media at appropriate concentration |

T Cell Culture Assay.

T cells were added to tissue culture wells coated with plate-bound anti-CD3 (8 μg/ml) plus either 3 μg/ml PD-L2Ig or control Ig fusion protein. Cells were added at 1×10⁵ cells/well in 100 μL of media. Drug or media control was added in 100 μL. Plates were incubated for up to 96 hours. Supernatants were harvested at various times for cytokine analyses. In some experiments, T cells were labeled with CFSE to assess T cell proliferation, as will be discussed below.

Cytokine Detection Assay.

BD Biosciences' Cytometric Bead Array (CBA): Mouse Th1/Th2 Cytokine Kit (Cat #551287) was used to measure Interleukin-2 (IL-2), Interleukin-4 (IL-4), Interleukin-5 (IL-5), Interferon-γ (IFN-γ), and Tumor Necrosis Factor (TNF). This kit makes use of fluorescence detection by flow cytometry to allow for detection of multiple analytes. 5 beads population with distinct fluorescent intensities are coated with specific antibodies for the cytokines. These beads are then mixed with a PE-conjugated detection reagent. The beads are incubated with the sample (supernatant from PD^{C} Tg or PD-1^{C} KO CD4 T cell culture) or a standard curve. The concentration of the unknown samples can then be extrapolated from the standard curve for each cytokine. The table below outlines this procedure.

| | |
|---|---|
| 1 | Prepare Standards |
| | Mix kit's lyophilized standard with 200 μL assay diluent being |
| | careful to mix without foaming, incubate @ RT for 15 min |
| 2 | Prepare highest standard curve concentration at 5000 pg/mL |
| | Add 20 μL of resuspended standard with 180 μL of assay diluent |
| | Serial dilute 1:2 for an 10 point standard curve including a 0 pg/ml |
| | concentration |
| 3 | Prepare beads |
| | Vortex each tube of bead gently but thoroughly |
| | Take 4 μL of each of the 5 beads (IL-2, IL-4, IL-5, IFN-γ, |
| | & TNF) for each sample and standard being measured and |
| | mix them into a tube for a “bead mixture” |
| 4 | Prepare samples |
| | Take 50 μL of supernatant from CD4 T cell cultures and transfer |
| | into a 96 well V-bottom plate |
| 5 | Reaction |
| | Add 20 μL of the bead mixture and 25 μL of the detection reagent |
| | into the 50 μL of supernatant in the V-bottom plate |
| | Incubate @ RT for 2 hrs |
| 6 | Wash 3× with Wash buffer flicking out the wash buffer from the |
| | plate each time |
| 7 | Add at least 50 μL of Wash buffer to the plate (more volume if |
| | not using HTS Flow cytometer) |
| 8 | Analyze samples using flow cytometer |
| 9 | Setting up the Flow Cytometer |
| | Prepare 3 tubes of setup beads A, B, C |
| | Add 50 μL of FITC positive control to tube B |
| | Add 50 μL of PE positive control to tube C |
| | Leave tube A for negative control |
| | Incubate tubes A, B, & C @ RT for 30 min |
| 10 | Open CBA Setup template on Flow Cytometer |
| | Follow instructions of template for adjusting gain and |
| | compensation controls |
| 11 | Switch to CBA Acquistion Template on Flow Cytometer |
| | Run standards and sample |
| 12 | Analysis |
| | Use CBA analysis template to analyze data |

Analysis of T Cell Expansion by CFSE Dye Dilution Assay.

The CFSE Proliferation assay uses a fluorescent dye to measure proliferation of cells. In this assay cells are stained with CFSE (Carboxyfluorescein succinimidyl ester). Each time a cell proliferates, the CFSE will be diluted among the daughter cells. Since the fluorescent intensity will be halved in each daughter cell, this method can be used to determine the number of times the cell population proliferates. PD-1^{C} Tg and PD-1 KO cells can be stained to determine proliferation of the CD4 T cells with anti-CD3 stimulation and inhibited by PD-L2Ig under different drug conditions. The table below outlines this procedure.

| | |
|---|---|
| 1 | CFSE labeling |
| | Isolate CD4 T cells |
| | Count cells & resuspend @ 10⁷ cells/ml in serum free media (RPMI) |
| | Add 0.1 μL of 10 mM CFSE per 10⁷ cells |
| | Incubate cells @ 37° C. for 15 min |
| | Add 5 mls of sterile FBS to cell suspension |
| | Spin down cells for 5 minutes @ 1250 rpm |
| | Discard supernatant |
| | Wash an addition 2X with CR10 media |
| | Resuspend cells at an appropriate concentration |
| 2 | Coat plates with 8 μg/mL of anti-CD3 & either 3 μg/mlL of |
| | PDL2 or control Ig |
| 3 | Plate CFSE labeled cells @ 1 × 10⁵ cells/well in 100 μL of media |
| 4 | Add 100 μL of drug or 100 μL of media for controls |
| 5 | Incubate plate for 96 hours (4 days) |
| 6 | Harvest plate by taking 100 μL of supernatant for other assays |
| | (e.g. cytokine analysis assays) and transferring to a V-bottom |
| | 96 well plate |
| | Do this gently to avoid disturbing cell on bottom of well |
| 7 | Pipette rest of the 100 μL vigorously to resuspend the lightly |
| | attached CD4 cells and transfer to V-bottom 96 well plate |
| 8 | Spin down V-bottom plate with cells for 15 minutes @ 1250 rpm |
| | Wash cells 2× in FACs buffer (2% FBS & 10 mM Sodium Azide) |
| 9 | Add master mix of FACs reagents to well |
| | Master mix: CD4 APC @ 1:50 & 7AAD @ 1:30 |
| | Incubate in refrigerator for at least 20 minutes |
| 10 | Wash cells 2× in FACs buffer |
| 11 | Resuspend cells in 50 μL and start reading on flow cytometer (LSRII) |

IFN-γ Detection Assays.

The Mouse IFN-γ Flex Set Assay is a kit from BD Biosciences (Cat #558296). Using the same principles described in Cytometric Bead Array (CBA): Mouse Th1/Th2 Cytokine Kit, this kit denoted by the “Flex Set” label is more focused on one analyte. In this case it is the measurement of IFN-γ. Biolegend's version of this assay was also used. The assays were compared to ensure continuity of data and Biolegend's protocol was adjusted to provide the same low level of background seen in the BD Biosciences Flex Set kit. The table below outlines this procedure.

| | |
|---|---|
| 1 | Coat plates with 8 μg/mL of anti-CD3 & either 3 μg/mL of |
| | PDL2 or control Ig |
| 2 | Plate cells @ 1 × 10⁵ cells/well in 100 μL of CR10 media |
| 3 | Add 100 μL of drug or 100 μL of CR10 media for controls |
| 4 | Incubate plate for 96 hours (4 days) |
| 5 | Harvest plate by taking 100 μL of supernatant for other assays and |
| | transferring to 2 separate V-bottom 96 well plate at 50 μl each |
| | Do this gently to avoid disturbing cell on bottom of well |
| 6 | Spin down V-bottom plate with cells for 15 minutes @ 1250 rpm |
| 7 | Prepare a IFN-γ standard curve |
| 8 | Add IFN-γ beads @ 0.25 μL of beads per well |
| | Incubate beads/supernatant mixture for 1 hour |
| 9 | Add detection reagent @ 0.25 μL of reagent per well |
| | Incubate beads/supernatant/detection reagent mixture for 1 hour |
| 10 | Wash mixture 2× in FACs buffer |
| 11 | Resuspend beads in 40 μL for 384 wells and 100 μL for |
| | 96 well plates |
| | Read on flow cytometer (LSRII) |

In each of these assays the goal was to measure the amount of IFN-γ that was present in the supernatant of CD4 T cells following incubation with drug or controls. These assays were used for both the large high-throughput screen of compounds and the follow up validation experiments.

IFN-γ ELISA Assay.

The IFN-γ ELISA is a sandwich ELISA based on an R& D systems IFN-γ kit. The kit included the capture and biotinylated detection antibody. A secondary antibody was still required for measurement of the levels of IFN-γ cytokine This assay used an anti-IFN-γ as the capture antibody. The IFN-γ in the supernatant of T cells (PD-1^{C} or PD-1 KO) incubated with and without drug would be incubated with the capture antibody. Then a detection antibody, anti-IFN-γ conjugated with biotin would be added to bind to the IFN-γ. Streptavidin conjugated to IR 800 Dye was used to visualize the interaction. Biotin on the detection antibody would bind to the streptavidin on the dye conjugate. The IR 800 Dye could then be detected by a plate reader.

### INCORPORATION BY REFERENCE

The following references correspond to the numbers above in parenthesis. All of these references are incorporated by reference in their entirety. In addition, all of the U.S. patents and U.S. published patent applications cited herein are hereby incorporated by reference.
1. Zajac, A. J., J. N. Blattman, K. Murali-Krishna, D. J. Sourdive, M. Suresh, J. D. Altman, and R. Ahmed. 1998. Viral immune evasion due to persistence of activated T cells without effector function. J Exp Med 188:2205-2213.
2. Matloubian, M., R. J. Concepcion, and R. Ahmed. 1994. CD4+ T cells are required to sustain CD8+ cytotoxic T-cell responses during chronic viral infection. J Virol 68:8056-8063.
3. Wherry, E. J., and R. Ahmed. 2004. Memory CD8 T-cell differentiation during viral infection. J Virol 78:5535-5545.
4. Wherry, E. J., J. N. Blattman, K. Murali-Krishna, R. van der Most, and R. Ahmed. 2003. Viral persistence alters CD8 T-cell immunodominance and tissue distribution and results in distinct stages of functional impairment. J Virol 77:4911-4927.
5. Betts, M. R., M. C. Nason, S. M. West, S. C. De Rosa, S. A. Migueles, J. Abraham, M. M. Lederman, J. M. Benito, P. A. Goepfert, M. Connors, M. Roederer, and R. A. Koup. 2006. HIV nonprogressors preferentially maintain highly functional HIV-specific CD8+ T cells. Blood 107:4781-4789.
6. Betts, M. R., B. Exley, D. A. Price, A. Bansal, Z. T. Camacho, V. Teaberry, S. M. West, D. R. Ambrozak, G. Tomaras, M. Roederer, J. M. Kilby, J. Tartaglia, R. Belshe, F. Gao, D. C. Douek, K. J. Weinhold, R. A. Koup, P. Goepfert, and G. Ferrari. 2005. Characterization of functional and phenotypic changes in anti-Gag vaccine-induced T cell responses and their role in protection after HIV-1 infection. Proc Natl Acad Sci USA 102:4512-4517.
7. Klenerman, P., and A. Hill. 2005. T cells and viral persistence: lessons from diverse infections. Nat Immunol 6:873-879.
8. Pentcheva-Hoang, T., E. Corse, and J. P. Allison. 2009. Negative regulators of T-cell activation: potential targets for therapeutic intervention in cancer, autoimmune disease, and persistent infections. Immunol Rev 229:67-87.
9. Nurieva, R. I., X. Liu, and C. Dong. 2009. Yin-Yang of costimulation: crucial controls of immune tolerance and function. Immunol Rev 229:88-100.
10. Sharpe, A. H. 2009. Mechanisms of costimulation. Immunol Rev 229:5-11.
11. Sharpe, A. H., E. J. Wherry, R. Ahmed, and G. J. Freeman. 2007. The function of programmed cell death 1 and its ligands in regulating autoimmunity and infection. Nat Immunol 8:239-245.
12. Okazaki, T., and T. Honjo. 2007. PD-1 and PD-1 ligands: from discovery to clinical application. Int Immunol 19:813-824.
13. Keir, M. E., M. J. Butte, G. J. Freeman, and A. H. Sharpe. 2008. PD-1 and its ligands in tolerance and immunity. Annu Rev Immunol 26:677-704.
14. Barber, D. L., E. J. Wherry, D. Masopust, B. Zhu, J. P. Allison, A. H. Sharpe, G. J. Freeman, and R. Ahmed. 2006. Restoring function in exhausted CD8 T cells during chronic viral infection. Nature 439:682-687.
15. Ha, S. J., S. N. Mueller, E. J. Wherry, D. L. Barber, R. D. Aubert, A. H. Sharpe, G. J. Freeman, and R. Ahmed. 2008. Enhancing therapeutic vaccination by blocking PD-1-mediated inhibitory signals during chronic infection. J Exp Med 205:543-555.
16. Day, C. L., D. E. Kaufmann, P. Kiepiela, J. A. Brown, E. S. Moodley, S. Reddy, E. W. Mackey, J. D. Miller, A. J. Leslie, C. DePierres, Z. Mncube, J. Duraiswamy, B. Zhu, Q. Eichbaum, M. Altfeld, E. J. Wherry, H. M. Coovadia, P. J. Goulder, P. Klenerman, R. Ahmed, G. J. Freeman, and B. D. Walker. 2006. PD-1 expression on HIV-specific T cells is associated with T-cell exhaustion and disease progression. Nature 443:350-354.
17. Urbani, S., B. Amadei, D. Tola, M. Massari, S. Schivazappa, G. Missale, and C. Ferrari. 2006. PD-1 expression in acute hepatitis C virus (HCV) infection is associated with HCV-specific CD8 exhaustion. J Virol 80:11398-11403.
18. Boettler, T., E. Panther, B. Bengsch, N. Nazarova, H. C. Spangenberg, H. E. Blum, and R. Thimme. 2006. Expression of the interleukin-7 receptor alpha chain (CD127) on virus-specific CD8+ T cells identifies functionally and phenotypically defined memory T cells during acute resolving hepatitis B virus infection. J Virol 80:3532-3540.
19. Trautmann, L., L. Janbazian, N. Chomont, E. A. Said, S. Gimmig, B. Bessette, M. R. Boulassel, E. Delwart, H. Sepulveda, R. S. Balderas, J. P. Routy, E. K. Haddad, and R. P. Sekaly. 2006. Upregulation of PD-1 expression on HIV-specific CD8(+) T cells leads to reversible immune dysfunction. Nat Med 12:1198-1202.
20. Petrovas, C., J. P. Casazza, J. M. Brenchley, D. A. Price, E. Gostick, W. C. Adams, M. L. Precopio, T. Schacker, M. Roederer, D. C. Douek, and R. A. Koup. 2006. PD-1 is a regulator of virus-specific CD8+ T cell survival in HIV infection. J Exp Med 203:2281-2292.
21. Radziewicz, H., C. C. Ibegbu, M. L. Fernandez, K. A. Workowski, K. Obideen, M. Wehbi, H. L. Hanson, J. P. Steinberg, D. Masopust, E. J. Wherry, J. D. Altman, B. T. Rouse, G. J. Freeman, R. Ahmed, and A. Grakoui. 2007. Liver-infiltrating lymphocytes in chronic human hepatitis C virus infection display an exhausted phenotype with high levels of PD-1 and low levels of CD127 expression. J Virol 81:2545-2553.
22. Freeman, G. J., E. J. Wherry, R. Ahmed, and A. H. Sharpe. 2006. Reinvigorating exhausted HIV-specific T cells via PD-1-PD-1 ligand blockade. J Exp Med 203:2223-2227.
23. Boni, C., P. Fisicaro, C. Valdatta, B. Amadei, P. Di Vincenzo, T. Giuberti, D. Laccabue, A. Zerbini, A. Cavalli, G. Missale, A. Bertoletti, and C. Ferrari. 2007. Characterization of hepatitis B virus (HBV)-specific T-cell dysfunction in chronic HBV infection. J Virol 81:4215-4225.
24. Chen, L., Z. Zhang, W. Chen, Z. Zhang, Y. L1, M. Shi, J. Zhang, L. Chen, S. Wang, and F. S. Wang. 2007. B7-H1 up-regulation on myeloid dendritic cells significantly suppresses T cell immune function in patients with chronic hepatitis B. J Immunol 178:6634-6641.
25. Zhang, J. Y., Z. Zhang, X. Wang, J. L. Fu, J. Yao, Y. Jiao, L. Chen, H. Zhang, J. Wei, L. Jin, M. Shi, G. F. Gao, H. Wu, and F. S. Wang. 2007. PD-1 up-regulation is correlated with HIV-specific memory CD8+ T-cell exhaustion in typical progressors but not in long-term nonprogressors. Blood 109:4671-4678.
26. Yao, Z. Q., E. King, D. Prayther, D. Yin, and J. Moorman. 2007. T cell dysfunction by hepatitis C virus core protein involves PD-1/PDL-1 signaling. Viral Immunol 20:276-287.
27. Jeong, H. Y., Y. J. Lee, S. K. Seo, S. W. Lee, S. J. Park, J. N. Lee, H. S. Sohn, S. Yao, L. Chen, and I. Choi. 2008. Blocking of monocyte-associated B7-H1 (CD274) enhances HCV-specific T cell immunity in chronic hepatitis C infection. J Leukoc Biol 83:755-764.
28. Geng, L., G. Jiang, Y. Fang, S. Dong, H. Xie, Y. Chen, M. Shen, and S. Zheng. 2006. B7-H1 expression is upregulated in peripheral blood CD14+ monocytes of patients with chronic hepatitis B virus infection, which correlates with higher serum IL-10 levels. J Viral Hepat 13:725-733.
29. Trabattoni, D., M. Saresella, M. Biasin, A. Boasso, L. Piacentini, P. Ferrante, H. Dong, R. Maserati, G. M. Shearer, L. Chen, and M. Clerici. 2003. B7-H1 is up-regulated in HIV infection and is a novel surrogate marker of disease progression. Blood 101:2514-2520.
30. Velu, V., S. Kannanganat, C. Ibegbu, L. Chemareddi, F. Villinger, G. J. Freeman, R. Ahmed, and R. R. Amara. 2007. Elevated expression levels of inhibitory receptor programmed death 1 on simian immunodeficiency virus-specific CD8 T cells during chronic infection but not after vaccination. J Virol 81:5819-5828.
31. Velu, V., K. Titanji, B. Zhu, S. Husain, A. Pladevega, L. Lai, T. H. Vanderford, L. Chemareddi, G. Silvestri, G. J. Freeman, R. Ahmed, and R. R. Amara. 2009. Enhancing SIV-specific immunity in vivo by PD-1 blockade. Nature 458:206-210.
32. Beswick, E. J., I. V. Pinchuk, S. Das, D. W. Powell, and V. E. Reyes. 2007. Expression of the programmed death ligand 1, B7-H1, on gastric epithelial cells after Helicobacter pylori exposure promotes development of CD4+ CD25+ FoxP3+ regulatory T cells. Infect Immun 75:4334-4341.
33. Das, S., G. Suarez, E. J. Beswick, J. C. Sierra, D. Y. Graham, and V. E. Reyes. 2006. Expression of B7-H1 on gastric epithelial cells: its potential role in regulating T cells during Helicobacter pylori infection. J Immunol 176:3000-3009.
34. Sheppard, K. A., L. J. Fitz, J. M. Lee, C. Benander, J. A. George, J. Wooters, Y. Qiu, J. M. Jussif, L. L. Carter, C. R. Wood, and D. Chaudhary. 2004. PD-1 inhibits T-cell receptor induced phosphorylation of the ZAP70/CD3zeta signalosome and downstream signaling to PKCtheta. FEBS Lett 574:37-41.
35. Parry, R. V., J. M. Chemnitz, K. A. Frauwirth, A. R. Lanfranco, I. Braunstein, S. V. Kobayashi, P. S. Linsley, C. B. Thompson, and J. L. Riley. 2005. CTLA-4 and PD-1 receptors inhibit T-cell activation by distinct mechanisms. Mol Cell Biol 25:9543-9553.
36. Latchman, Y., C. R. Wood, T. Chernova, D. Chaudhary, M. Borde, I. Chernova, Y. Iwai, A. J. Long, J. A. Brown, R. Nunes, E. A. Greenfield, K. Bourque, V. A. Boussiotis, L. L. Carter, B. M. Carreno, N. Malenkovich, H. Nishimura, T. Okazaki, T. Honjo, A. H. Sharpe, and G. J. Freeman. 2001. PD-L2 is a second ligand for PD-1 and inhibits T cell activation. Nat Immunol 2:261-268.
37. Freeman, G. J., A. J. Long, Y. Iwai, K. Bourque, T. Chernova, H. Nishimura, L. J. Fitz, N. Malenkovich, T. Okazaki, M. C. Byrne, H. F. Horton, L. Fouser, L. Carter, V. Ling, M. R. Bowman, B. M. Carreno, M. Collins, C. R. Wood, and T. Honjo. 2000. Engagement of the PD-1 immunoinhibitory receptor by a novel B7 family member leads to negative regulation of lymphocyte activation. J Exp Med 192:1027-1034.
38. Riley, J. L. 2009. PD-1 signaling in primary T cells. Immunol Rev 229:114-125.
39. Chemnitz, J. M., R. V. Parry, K. E. Nichols, C. H. June, and J. L. Riley. 2004. SHP-1 and SHP-2 associate with immunoreceptor tyrosine-based switch motif of programmed death 1 upon primary human T cell stimulation, but only receptor ligation prevents T cell activation. J Immunol 173:945-954.
40. Keir, M. E., S. C. Liang, I. Guleria, Y. E. Latchman, A. Qipo, L. A. Albacker, M. Koulmanda, G. J. Freeman, M. H. Sayegh, and A. H. Sharpe. 2006. Tissue expression of PD-L1 mediates peripheral T cell tolerance. J Exp Med 203:883-895.
41. Carter, L., L. A. Fouser, J. Jussif, L. Fitz, B. Deng, C. R. Wood, M. Collins, T. Honjo, G. J. Freeman, and B. M. Carreno. 2002. PD-1:PD-L inhibitory pathway affects both CD4(+) and CD8(+) T cells and is overcome by IL-2. Eur J Immunol 32:634-643.
42. Wherry, E. J., J. N. Blattman, and R. Ahmed. 2005. Low CD8 T-cell proliferative potential and high viral load limit the effectiveness of therapeutic vaccination. J Virol 79:8960-8968.
43. Keir, M. E., Y. E. Latchman, G. J. Freeman, and A. H. Sharpe. 2005. Programmed death-1 (PD-1):PD-ligand 1 interactions inhibit TCR-mediated positive selection of thymocytes. J Immunol 175:7372-7379.
44. Keir, M. E., G. J. Freeman, and A. Sharpe. 2007. PD-1 regulates self-reactive CD8+ T cell responses to antigen in lymph nodes and tissues. Journal of Immunology 179:5064-5070.
45. Ansari, M. J. I., Salama, A. D., Chitnis, T., Smith, R. N., Yagita, H., Akiba, H., Yamazaki, T., Azuma, M., Iwai, J., Khoury, S. J., Auchincloss Jr, H., & Sayegh, M. H. (2003). The programmed death-1 (PD-1) pathway regulated autoimmune diabetes in nonobese diabetic (NOD) mice. J Exp Med, 198(1), 63-69.
46. Zhang, L., T. F. Gajewskil, and J. Kline (2009). PD-1/PD-L1 interactions inhibit antitumor immune responses in a murine acute myeloid leukemia model. Blood, 114(8), 1545-1552; and references to solid tumors cited therein.
47. Hirata S, Senju S, Matsuyoshi H, Fukuma D, Uemura Y, Nishimura Y. 2005. Prevention of experimental autoimmune encephalomyelitis by transfer of embryonic stem cell-derived dendritic cells expressing myelin oligodendrocyte glycoprotein peptide along with TRAIL or programmed death-1 ligand. J Immunol 174: 1888-97

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A compound of formula I represented by: 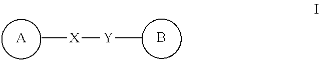
or a pharmaceutically acceptable salt, solvate, enantiomer or stereoisomer thereof,
wherein independently for each occurrence,
A is aryl, heteroaryl or biaryl;
B is aryl, heteroaryl or biaryl;
X is —N(R)—, —C(R)₂—, —O— or —S—;
R is hydrogen or alkyl; and
Y is —S(═O)₂—, —S(═O)— or —C(═O)—.

2. The compound of claim 1, wherein X is —N(R)—.

3. The compound of claim 1, wherein X is —N(H)—.

4. The compound of claim 1, wherein X is —N(CH₃)—.

5-7. (canceled)

8. The compound of claim 1, wherein Y is —S(═O)₂—.

9. (canceled)

10. The compound of claim 1, wherein Y is —C(═O)—.

11-13. (canceled)

14. The compound of claim 1, wherein A is 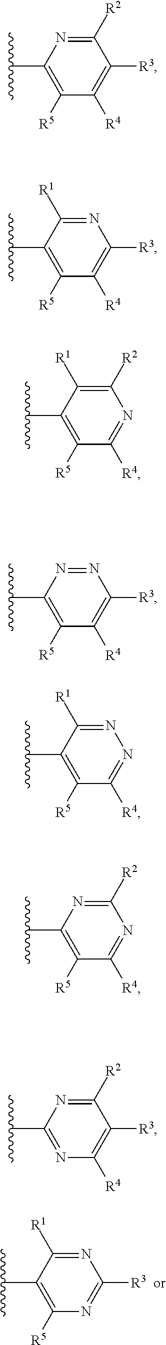 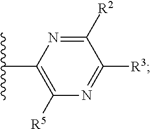
R¹ to R⁵ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

15-29. (canceled)

30. The compound of claim 1, wherein A is

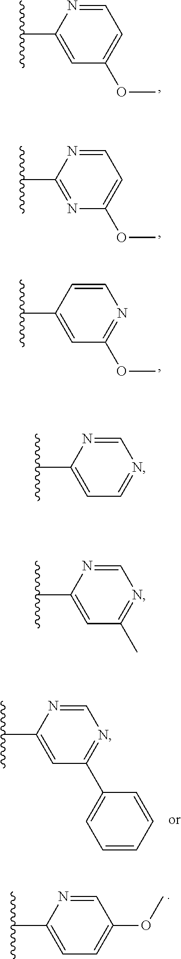

31. The compound of claim 1, wherein A is 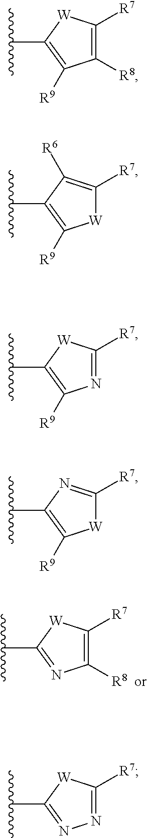
W is —S—, —O— or)-N(R¹⁰)—; R⁶ to R⁹ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹⁰ is hydrogen or alkyl; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

32-45. (canceled)

46. The compound of claim 1, wherein A is

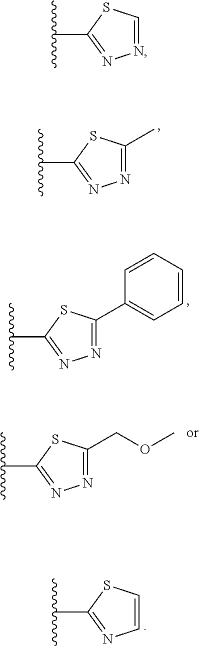

47-48. (canceled)

49. The compound of claim 1 wherein B is 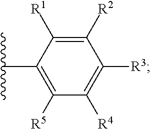
R¹ to R⁵ are independently selected from the group consisting of hydrogen, alkyl, heterocyclyl, aryl, heteroaryl, heterocyclylalkyl, aralkyl, heteroaralkyl, halo, haloalkyl, cyano, nitro, —N(R¹²)R¹³, —CH₂N(R¹²)R¹³, —CH₂CH₂N(R¹²)R¹³, —CH₂CH₂CH₂N(R¹²)R¹³, —OR¹², —CH₂OR¹², —CH₂CH₂OR¹² and —CH₂CH₂CH₂OR¹²; R¹⁰ is hydrogen or alkyl; R¹² is hydrogen, alkyl, haloalkyl, heterocyclyl, aryl, aralkyl, heteroaryl, heterocyclylalkyl, aralkyl, heteroaralkyl, formyl, alkylcarbonyl, haloalkylcarbonyl, heterocyclylcarbonyl, arylcarbonyl, aralkylcarbonyl, heteroarylcarbonyl, heterocyclylalkylcarbonyl, aralkylcarbonyl, heteroaralkylcarbonyl, alkyloxycarbonyl, haloalkyloxycarbonyl, heterocyclyloxycarbonyl, aryloxycarbonyl, aralkyloxycarbonyl, heteroaryloxycarbonyl, heterocyclylalkyloxycarbonyl, aralkyloxycarbonyl, heteroaralkyloxycarbonyl and amido; and R¹³ is hydrogen or alkyl.

50-73. (canceled)

74. The compound of claim 1 wherein B is 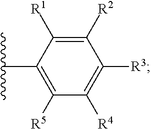
R¹, R², R⁴ and R⁵ are hydrogen; R³ is

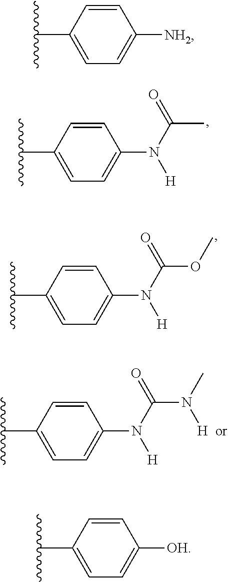

75. A compound, or a pharmaceutically acceptable salt, solvate, enantiomer or stereoisomer thereof, selected from the group consisting of

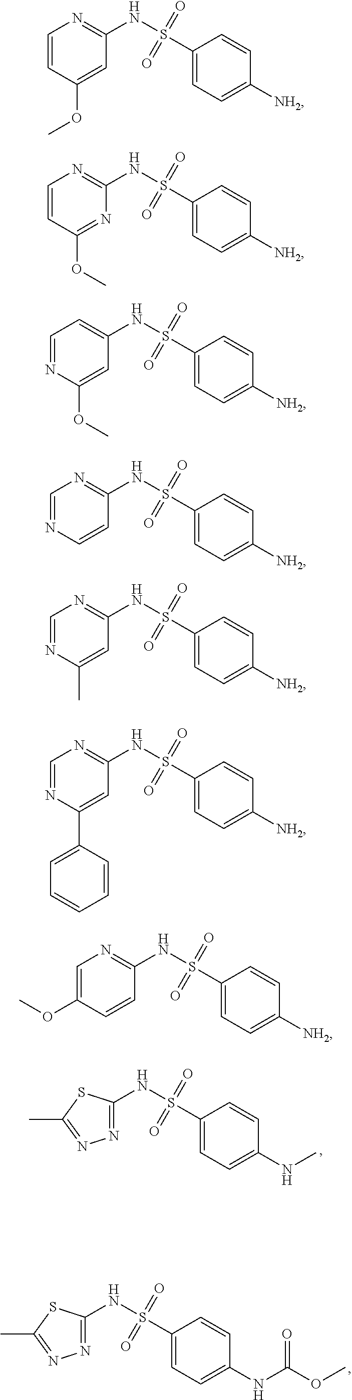

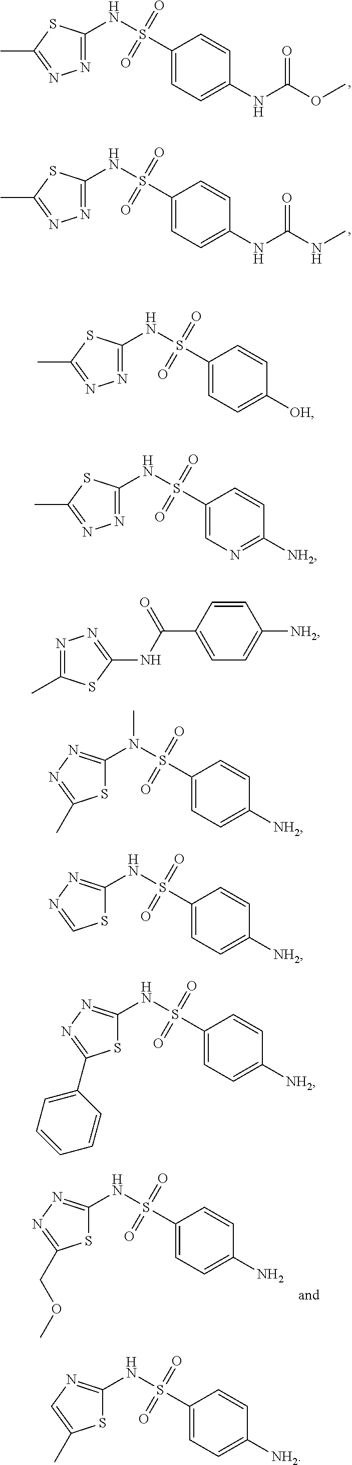

76-78. (canceled)

79. A pharmacutical composition comprising a compound of claim 1, and a pharmacutically acceptable excipient.

80. A method of treating or preventing a disorder in a subject comprising administering to the subject a therapeutically effective amount of a compound of claim 1, or a pharmaceutical composition of claim 79, to the subject.

81. The method of claim 80, wherein the disorder is an infectious disease, cancer, or an immune disorder.

82-89. (canceled)

90. A method for suppressing, treating, or preventing graft rejection accompanying the transplantation of an organ, or a portion thereof, or a tissue in a subject, the method comprising administering a therapeutically effective amount of a compound of claim 1, or a pharmaceutical composition of claim 79, to the subject.

91. The method of claim 90, wherein the transplantation is allotransplantation or xenotransplantation.

92-94. (canceled)

95. A method of enhancing an immune response to an antigen in a subject, comprising administering to the subject: (i) the antigen; and (ii) a compound of claim 1, or a pharmaceutical composition of claim 79, in an amount sufficient to enhance the subject's immune response to the antigen.

96. (canceled)
